(19) United States
(12) Patent Application Publication
(10) Pub. No.: US 2004/0137004 A1
(43) Pub. Date: Jul. 15, 2004

(54) Patch for transcutaneous immunization

(76) Inventors: Gregory M Glenn, Poolesville, MD (US); Jianmei Yu, Bethesda, MD (US); Mervyn L Hamer, Gaithersburg, MD (US); Jesus Miranda, Miami, FL (US); Christopher L Adams, Miramar, FL (US)

Correspondence Address: MORGAN LEWIS & BOCKIUS LLP, 1111 PENNSYLVANIA AVENUE NW, WASHINGTON, DC, 20004, US

(21) Appl. No.: 10/472,598

(22) PCT Filed: Mar. 19, 2002
(86) PCT No.: PCT/US02/08099

(51) Int. Cl. A61K 39/00; A61K 39/38

(52) U.S. Cl. 424/184.1

(57) ABSTRACT

A protein-in-adhesive patch for transcutaneous immunization is described with at least four different components: (i) backing layer; (ii) pressure-sensitive adhesive adhering to the backing layer; (iii) at last one immunologically-active protein of an immunogenic formulation applied to the pressure-sensitive adhesive layer opposite the backing layer and/or incorporated in the pressure-sensitive adhesive layer such that the at least one protein is in contact with adhesive; and (iv) stabilizer which maintains the immunological activity of the at least one protein under ambient conditions.

### CROSS-REFERENCE TO RELATED APPLICATIONS

This application claims the benefit of provisional U.S. Appln. No. 60/276,497, filed Mar. 19, 2001.

### FIELD OF THE INVENTION

The invention relates to a protein-in-adhesive patch for transcutaneous immunization, their use to treat disease, and their manufacture.

### BACKGROUND OF THE INVENTION

A variety of antigens are effectively administered by transcutaneous immunization (TCI) to induce antigen-specific immune responses. See WO 98/20734, WO 99/43350, and WO 00/61184; U.S. Pat. Nos. 5,910,306 and 5,980,898; and U.S. patent application Ser. Nos. 09/257,188; 09/309,881; 09/311,720; 09/316,069; 09/337,746; and 09/545,417. The immune response may require the use of an adjuvant (e.g., ADP-ribosylating exotoxins). Vaccines are safe and effective when applied epicutaneously, in contrast to the disadvantages associated with the use of some adjuvants when administered by an enteral, mucosal, transdermal or other parenteral route (e.g., subcutaneous, intramuscular, intraperitoneal, intraarterial, intravenous). Skin antigen presenting cells can be activated and antigen processed without eliciting undesirable immune reactions (e.g., atopy, dermatitis, eczema, psoriasis, and other allergic or hypersensitivity reactions). Here, we describe patches for transcutaneous immunization in which protein antigen is incorporated into an adhesive component in contact with skin of the human or animal to be immunized.

Drug-in-adhesive patches have been described, but most of them are limited to the transdermal administration of small molecular weight drugs (e.g., androgens, nicotine, nitroglycerin) to be introduced into the systemic circulation. But the incorporation of proteins, which are much larger than the aforementioned drugs and more unstable in their chemical and physical structure, into an adhesive portion of a patch for transcutaneous immunization has not been described. Proteinaceous adjuvants and antigens are subject to denaturation and degradation. Herein, we show that both a patch according to the present invention and its immunogenic proteins are mechanically and chemically stable, respectively. Moreover, biological activity of the immunogenic protein is maintained and microbial contamination is avoided even after prolonged storage at room temperature.

Protein-in-adhesive patches used for transcutaneous immunization, as well as processes for making and using them, are disclosed herein. In particular, the stability of protein in this formulation and use of the patch in TCI are demonstrated. Use of one or more stabilizers may avoid protein aggregation, degradation, denaturation, or combinations thereof. Other advantages of the invention are discussed below or would be apparent from the disclosure herein.

### SUMMARY OF THE INVENTION

A protein-in-adhesive patch for transcutaneous immunization is comprised of at least four different components: (i) backing layer; (ii) pressure-sensitive adhesive layer adhering to the backing layer; (iii) at least one immunologically-active protein of an immunogenic formulation applied to the pressure-sensitive adhesive layer opposite the backing layer and/or incorporated in the pressure-sensitive adhesive layer such that the at least one protein is in contact with adhesive; and (iv) stabilizer which maintains the immunological activity of the at least one protein under ambient conditions.

The backing layer may be occlusive or semi-occlusive (e.g., dressing). An optional release liner may be included. A single unit may be produced by enclosing a patch in packaging material sufficient for storage under ambient conditions.

The pressure-sensitive adhesive layer may be comprised of at least one aqueous-based adhesive (e.g., acrylate or silicone). The stabilizer may be a sugar or polymer to protect the protein: for example, a nonreducing disaccharide, sucrose, or trehalose may be used. Other excipients such as plasticizer, tackifier, and thickener may be included in a formulation containing adhesive, adjuvant, or antigen. The plasticizer may be a short-chain trialkyl citrate. The tackifier may be a glycol and/or succinic acid. The thickener may be a short-chain hydroxyalkyl cellulose or starch.

The protein may have adjuvant activity, antigen activity, or both. The protein may be the antigen against which the immune response is induced or it may act as adjuvant to promote the immune response induced by a heterologous antigen. An ADP-ribosylating exotoxin (e.g., cholera toxin, diphtheria toxin, E coli heat-labile enterotoxin, Pseudomonas exotoxin A, pertussis toxin), a chemokine, a cytokine, other known adjuvants, or derivatives thereof may be the protein. Examples of the derivatives are fragments (including those that have been chemically conjugated or genetically fused with a portion of the wild-type adjuvant) or mutants (including those that are naturally occurring variants or other changes, insertions, or deletions in the amino acid sequence) which have adjuvant activity.

An effective amount of the protein is provided by the patch. For example, the patch may comprise an amount of protein between 1 μg and 1 mg, 5 μg and 500 μg, 10 μg and 100 μg, or intermediate ranges thereof. Depending on the immunologic activity of the protein, the effective amount of a particular protein may vary.

Transcutaneous immunization may be used for inducing an antigen-specific immune respone, treating an existing disease, or preventing a disease for which the subject is at risk. Hydration or penetration of the skin at the site where the patch is used may enhance the antigen-specific immune response or prevent unwanted immune reactions. A possible target for activation and/or presentation of antigen is a dendritic cell underlying the skin.

A wet blend may be formulated containing adhesive and stabilized protein, and then used to manufacture a patch. Protein may be applied to the surface of an adhesive layer, incorporated in an adhesive as a suspension or in solution, or the adhesive- and protein-containing formulations may be separately made and then mixed or laminated together. Casting, coating, extrusion, laminating, and printing may be used to bring protein in contact with adhesive.

Effectiveness may be assessed by one or more clinical or laboratory criteria, surrogate markers which are correlated to health, or morbidity or mortality criteria. Further aspects of the invention will be apparent to a person skilled in the art from the following detailed description and claims, and generalizations thereto.

### DESCRIPTION OF THE DRAWINGS

FIG. 1 is a schematic, partial cross-section of a protein-in-adhesive (PIA) patch 10. A backing layer 12 and a pressure-sensitive adhesive layer 14 adhere to each other. The skin-side of the patch 10 is optionally covered prior to use by a release liner 18. An immunogenic formulation 16 is located on the exposed side of the patch 10 by application to the skin-side of the pressure-sensitive adhesive layer 14. It is not necessarily shown to scale.

FIG. 2 is a schematic, partial cross-section of a protein-in-adhesive (PIA) patch 20. A backing layer 22 and a pressure-sensitive adhesive layer 24 adhere to each other. The skin-side of the patch 20 is optionally covered prior to use by a release liner 28. An immunogenic formulation 26 is located on the exposed side of the patch 20 by incorporation in the pressure-sensitive adhesive layer 24. It is not necessarily shown to scale.

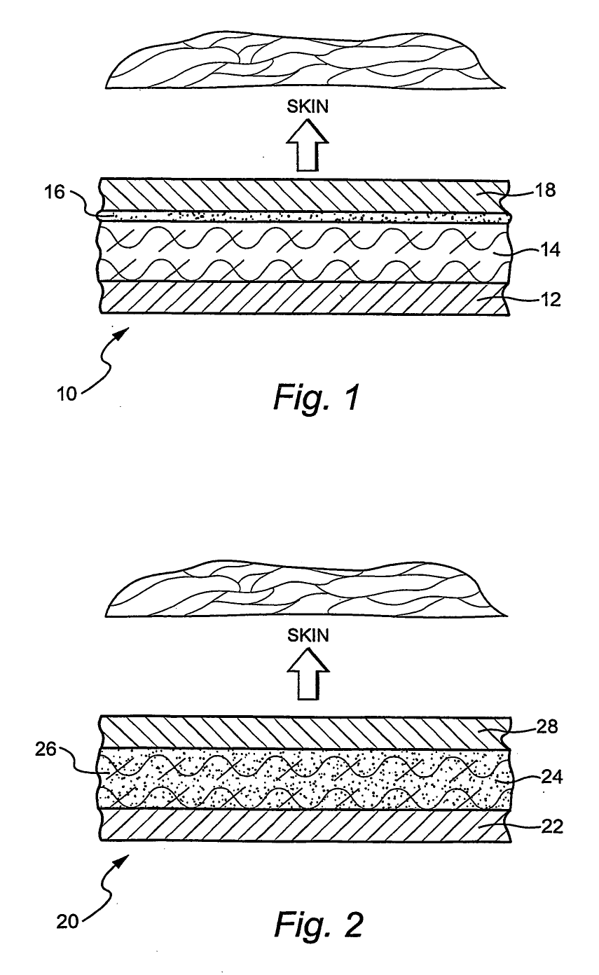

### DESCRIPTION OF SPECIFIC EMBODIMENTS OF THE INVENTION

One or more active immunogenic proteins used in transcutaneous immunization may be applied to and/or incorporated in an adhesive portion of a patch or an adhesive formulation per se by dispersing or solubilizing proteins with a stabilizer under nondenaturing conditions. In contrast to a syrup or other sticky solution, the adhesive is pressure sensitive. The protein-containing immunogenic formulation is stabilized against degradation and loss of adjuvant and/or antigen activity. If not it is not soluble in an aqueous adhesive, the stabilized protein or particles containing the stabilized protein may be added as a slurry or suspension. We have termed this a “protein-in-adhesive” (PIA) patch.

Formulations typically used for drug-in-adhesive (DIA) products are unacceptable for proteins as the solubilization of the adhesive in DIA was usually performed in an organic solvent which was thought to be unsuitable for unprotected proteins. By changing the base of the adhesive to an aqueous-based adhesive formulation and incorporating proteins into the water-based adhesive formulation, the active ingredients for transcutaneous immunization can be incorporated into an adhesive portion of a patch or an adhesive formulation per se.

The DIA concept has been used in transdermal drug delivery, which is distinguished from transcutaneous immunization by several features (Glenn et al., Exp. Opin. Invest. Drugs, 8:797-805, 1999). Thus, use of PIA formulations and patches for transcutaneous immunization represents a new and nonobvious invention.

The adhesive may be used for one or more of the following purposes: to keep the patch in place on the subject, to incorporate other components of the formulation such as optional skin penetration enhancer chemicals or non-active components, to stabilize labile components of the formulation, and other purposes known to skilled artisans. The use of adhesive patches for purposes of transcutaneous immunization provides a convenient and practical method for administration of vaccine.

Skin Structure and Immunobiology

Skin, the largest human organ, plays an important part in the body's defense against invasion by infectious agents and contact with noxious substances. But this barrier function of the skin appears to have prevented the art from appreciating that transcutaneous immunization provided an effective alternative to enteral, mucosal, and other parenteral routes of administering vaccines. It has recently been shown that epicutaneous application of a vaccine targets specialized antigen presenting cells and induces a robust immune response.

Anatomically, skin is composed of three layers: the epidermis, the dermis, and subcutaneous fat. Epidermis is composed of the basal, the spinous, the granular, and the cornified layers; the stratum corneum comprises the cornified layer and lipid. The principal antigen presenting cells of the skin, Langerhans cells, are reported to be in the mid- to upper-spinous layers of the epidermis in humans. Dermis contains primarily connective tissue. Blood and lymphatic vessels are confined to the dermis and subcutaneous fat.

The stratum corneum, a layer of dead skin cells and lipids, has traditionally been viewed as a barrier to the hostile world, excluding organisms and noxious substances from the viable cells below the stratum corneum. Stratum corneum also serves as a barrier to the loss of moisture from the skin: the relatively dry stratum corneum is reported to have 5% to 15% water content while deeper epidermal and dermal layers are relatively well hydrated with 85% to 90% water content. The barrier function of skin is reinforced by extensive crosslinking between corneocytes. Only recently has the secondary protection provided by antigen presenting cells (e.g., Langerhans cells) been recognized. Moreover, the ability to immunize through the skin with or without penetration enhancement (i.e., transcutaneous immunization) using a skin-active adjuvant has only been recently described. Although undesirable skin reactions such as atopy and dermatitis were known in the art, recognition of the therapeutic advantages of transcutaneous immunization might not have been appreciated in the past because the skin was believed to provide a barrier to the passage of molecules larger than about 500 daltons (Bos et al., Exp. Dermatol., 9:165-169, 2000).

The epidermis is composed primarily of keratinocytes, but also has a significant population (about 1% to 3%) of immune surveillance cells called Langerhans cells (LC) distributed amongst the viable keratinocytes. Although LC are a relatively small population of cells in the skin, they account for 25% of the total skin surface area in humans. Langerhans cells represent an extensive, superficial network barrier of immune cells that make an attractive target for vaccine delivery. They are bone marrow derived dendritic cells that migrate to epithelial surfaces where they perform immunosurveilance. Under normal circumstances, there is a baseline traffic of LC from the skin to the draining lymph nodes. In the face of a stimulus such as infecting microbes, the number of LC migrating out of the skin is greatly increased, fulfilling the immunosurveillance function of an antigen presenting cell. Langerhans cells stimulated by the danger signals created by interaction with microbes, foreign materials, or adjuvants orchestrate an effector immune response in the lymph node through the highly specific and amplified response created by their-antigen presentation function.

A system for transcutaneous immunization (TCI) is provided which induces an immune response (e.g., humoral and/or cellular effector specific for an antigen) in a human or animal. The delivery system provides simple, epicutaneous application of a formulation comprised of at least one adjuvant and one or more antigens to the skin of a human or animal subject (Glenn et al., J. Immunol., 161:3211-3214, 1998a; Glenn et al., Nature, 391:851, 1998b; Glenn et al., Nature Med., 6:1403-1406, 2000; Hammond et al., Adv. Drug Deliv. Rev., 43:45-55, 2000; Scharton-Kersten et al., Infect. Immun., 68:5306-5313, 2000). An antigen-specific immune response is thereby induced with or without chemical and/or physical penetration enhancement as long as the skin is not perforated through the dermal layer. This delivery system may also be used in conjunction with enteral, mucosal, or other parenteral immunization techniques. Thus, the patch technologies described here could be used for treatment of humans and animals such as, for example, immunotherapy and immunoprotection: therapeutically to treat existing disease, protectively to prevent disease, to reduce the severity and/or duration of disease, to ameliorate one or more symptoms of disease, or combinations thereof.

The transit pathways utilized by antigens to traverse the stratum corneum are unknown at this time. The stratum corneum (SC) is the principal barrier to delivery of drugs and antigens through the skin. Transdermal drug delivery of polar drugs is widely held to occur through aqueous intercellular channels formed between the keratinocytes (Transdermal and Topical Drug Delivery Systems, Eds. Ghosh et al., Buffalo Grove: Interpharm Press, 1997). Although the SC is the limiting barrier for penetration, it is breached by hair follicles and sweat ducts. Whether antigens penetrate directly through the SC or via the epidermal appendages may depend on a host of factors. These-appendages-are thought to play only a minor role in transdermal drug delivery (Barry et al., J. Control Rel., 6:85-97, 1987). Despite some evidence in mice that transcutaneous immunization using DNA may utilize hair follicles as the pathway for skin penetration (Fan et al., Nature Biotechnol., 17:870-872, 1999), it is more likely that the robust immune responses utilize more of the skin surface area. Because disruption of the SC barrier can be accomplished by simple hydration of the skin (Roberts et al., In: Pharmaceutical Skin Penetration Enhancement, Eds. Walters et al., New York: Marcel Dekker, 1993), this has been employed for transcutaneous immunization.

Activation of one or more of adjuvant, antigen, and antigen presenting cell (APC) may promote the induction of the immune response. The APC processes the antigen and then presents one or more epitopes to a lymphocyte. Activation may promote contact between the formulation and the APC (e.g., Langerhans cells, other dendritic cells, macrophages, B lymphocytes), uptake of the formulation by the APC, processing of antigen and/or presentation of epitopes by the APC, migration and/or differentiation of the APC, interaction between the APC and the lymphocyte, or combinations thereof. The adjuvant by itself may activate the APC. For example, a chemokine may recruit and/or activate antigen presenting cells to a site. In particular, the antigen presenting cell may migrate from the skin to the lymph nodes, and then present antigen to a lymphocyte, thereby inducing an antigen-specific immune response. Furthermore, the formulation may directly contact a lymphocyte which recognizes antigen, thereby inducing an antigen-specific immune response.

In addition to eliciting immune reactions leading to activation and/or expansion of antigen-specific B-cell and/or T-cell populations, including antibodies and cytotoxic T lymphocytes (CTL), the invention may positively and/or negatively regulate one or more components of the immune system by using transcutaneous immunization to affect antigen-specific helper (Th1 and/or Th2) or delayed-type hypersensitivity T-cell subsets (T_{DTH}). The desired immune response induced is preferably systemic or regional (e.g., mucosal) but it is usually not undesirable immune responses (e.g., atopy, dermatitis, eczema, psoriasis, and other allergic or hypersensitivity reactions). As seen herein, the immune responses induced are of the quantity and quality that provide therapeutic or prophylactic immune responses useful for treating disease.

Hydration of the intact or penetrated skin before, during, or immediately after epicutaneous application of the formulation is preferred and may be required in some or many instances. For example, hydration may increase the water content of the topmost layer of skin (e.g., stratum corneum or superficial epidermis layer exposed by penetration enhancement techniques) above 25%, 50% or 75%. Skin may be hydrated with an aqueous solution of 10% glycerol, 70% isopropyl alcohol, and 20% water. Addition of an occlusive dressing or use of a semi-liquid formulation (e.g., cream, emulsion, gel, lotion, paste) can increase hydration of the skin. For example, lipid vesicles or sugars can be added to a formulation to thicken a solution or suspension. Hydration occurs with or without disruption of all or at least a portion of the stratum corneum at the site of application of the formulation, along with possibly also a portion of the epidermis, as long as the dermis is not perforated. The intent is for the formulation to act on skin antigen presenting cells instead of introducing immunologically-active components of the formulation into the systemic circulation, although some portion of the formulation may act at distal sites.

Skin may be swabbed with an applicator (e.g., adsorbent material on a pad or stick) containing hydration or chemical penetration agents or they may be applied directly to skin. For example, aqueous solutions (e.g., water, saline, other buffers), acetone, alcohols (e.g., isopropyl alcohol), detergents (e.g., sodium dodecyl sulfate), depilatory or keratinolytic agents (e.g., calcium hydroxide, salicylic acid, ureas), humectants (e.g., glycerol, other glycols), polymers (e.g., polyethylene or propylene glycol, polyvinyl pyrrolidone), or combinations thereof may be used or incorporated in the formulation. Similarly, abrading the skin (e.g., abrasives like an emery board or paper, sand paper, fibrous pad, pumice), removing a superficial layer of skin (e.g., peeling or stripping with an adhesive tape), microporating the skin using an energy source (e.g., heat, light, sound, electrical, magnetic) or a barrier disruption device (e.g., blade, needle, projectile, spray, tine), or combinations thereof may act as a physical penetration enhancer. See WO 98/29134, WO 01/34185, and WO 02/07813; U.S. Pat. Nos. 5,445,611, 6,090,790, 6,142,939, 6,168,587, 6,312,612, 6,322,808 and 6,334,856 for description of microblades or microneedles, gun or spray injectors, and for microporation of the skin and techniques that might be adapted for transcutaneous immunization. The objective of chemical or physical penetration enhancement in conjunction with TCI is to remove at least the stratum corneum, or a superficial or deeper epidermal layer, without perforating skin through past the dermal layer. This is preferably accomplished with minor discomfort at most to the human or animal subject and without bleeding at the site. For example, applying the formulation to intact skin may or may not involve thermal, optical, sonic or electromagnetic energy to perforate layers of the skin to below the stratum corneum or epidermis.

The difference between transcutaneous immunization as practiced in WO 98/20734 and 99/43350 is whether all or at least a portion of the stratum corneum is disrupted. The term “penetration enhancer” as used herein refers to those chemicals which when applied in the formulation, before application, during application, or after application results in such disruption. Some chemicals (e.g., alcohols) may or may not disrupt the stratum corneum depending on how vigorously they are applied (e.g., swabbing or scrubbing with sufficient pressure). For example, including alcohol, O/W or W/O emulsions, lipid micelles, or lipid vesicles in the formulation may enhance penetration of one or more immunologically-active ingredients of the same formulation across intact skin without detectable disruption of the stratum corneum.

Formulations which are useful for vaccination are also provided as well as processes for their manufacture. The formulation may be in liquid or semi-liquid form. For example, the formulation may be provided as a liquid: cream, emulsion, gel, lotion, ointment, paste, solution, suspension, or other liquid forms. Formulation may be air dried, dried with elevated temperature, freeze or spray dried, coated or sprayed on a solid substrate and then dried, dusted on a solid substrate, quickly frozen and then slowly dried under vacuum, or combinations thereof to a low moisture content. Adhesive formulations may be cured to a desired amount of crosslinking by suitable choice of initiator, rate accelerator or decelerator, and terminator.

A “patch” refers to a product which includes a solid substrate (e.g., occlusive or nonocclusive surgical dressing) as well as at least one active ingredient. Liquid or semi-liquid formulations may be incorporated in a patch. Here, the patch comprises backing layer, pressure-sensitive adhesive layer, and immunogenic formulation. The solid substrate is at least the backing layer, but the adhesive and immunogenic formulations may also form part of the solid substrate is they are suitably dried and cured. One or more active components of the immunogenic formulation may be applied on the adhesive layer, incorporated in the adhesive layer, or combinations thereof. Layers may be formed, and then adhered or laminated together.

The moisture content of the adhesive layer may be more than 0.5%, more than 1%, more than 2%, less than 10%, less than 5%, less than 2%, and intermediate ranges thereof. The patch may be a pliable, planar substrate from about 1 cm² to about 100 cm². An effective amount of the protein is provided by a single patch. For example, the patch may comprise an amount of protein between 1 μg and 1 mg, 5 μg and 500 μg, 10 μg and 100 μg, or intermediate ranges thereof. Depending on the immunologic activity of the protein, the effective amount of a particular protein may vary. The patch may be stored in a moisture-proof package (e.g., blister pack, foil pouch) for at least one or two years at room temperature (e.g., 20° C. to 30° C.) with an immunological activity between 85% and 115% of the patch's initial activity.

Formulation in liquid or semi-liquid form may be applied with one or more adjuvants and/or antigens both at the same or separate sites or simultaneously or in frequent, repeated applications. The patch may include a controlled-release reservoir or a rate-controlling matrix or membrane may be used which allows stepped release of adjuvant and/or antigen. It may contain a single reservoir with adjuvant and/or antigen, or multiple reservoirs to separate individual antigens and adjuvants. The patch may include additional antigens such that application of the patch induces an immune response to multiple antigens. In such a case, antigens may or may not be derived from the same source, but they will have different chemical structures so as to induce an immune response specific for different antigens. Multiple patches may be applied simultaneously; a single patch may contain multiple reservoirs. For effective treatment, multiple patches may be applied at intervals or constantly over a period of time; they may be applied at different times, for overlapping periods, or simultaneously.

Solids (e.g., particles of nanometer or micrometer dimensions) may also be incorporated in the formulation. Solid forms (e.g., nanoparticles or microparticles) may aid in dispersion or solubilization of active ingredients; assist in carrying the formulation through superficial layers of the skin; provide a point of attachment for adjuvant, antigen, or both to a substrate that can be opsonized by antigen presenting cells, or combinations thereof. Ingredients that are insoluble or poorly soluble in an aqueous solution may be formulated in an emulsion, lipid vesicles, or micelles.

The formulation may be manufactured under conditions acceptable to appropriate regulatory agencies (e.g., Food and Drug Administration) for biologicals and vaccines. Optionally, components like binders, buffers, colorings, dessicants, diluents, humectants, preservatives, stabilizers, other excipients, adhesives, plasticizers, tackifiers, thickeners, patch materials, or combinations thereof may be included in the formulation even though they are immunologically inactive. They may, however, have other desirable properties or characteristics which improve the effectiveness of the formulation.

A single or unit dose of formulation suitable for administration is provided. The amount of adjuvant or antigen in the unit dose may be anywhere in a broad range from about 0.001 μg to about 10 mg. This range may be from about 0.1 μg to about 1 mg; a narrower range is from about 5 μg to about 500 μg. Other suitable ranges are between about 1 μg and about 10 μg, between about 10 μg and about 50 μg, between about 50 μg and about 200 μg, and between about 1 mg and about 5 mg. A preferred dose for a toxin is about 50 μg or 100 μg or less (e.g., from about 1 μg to about 50 μg or 100 μg). The ratio between antigen and adjuvant may be about 1:1 (e.g., an ADP-ribosylating exotoxin when it is both antigen and adjuvant) but higher ratios may be suitable for poor antigens (e.g., about 1:10 or less), or lower ratios of antigen to adjuvant may also be used (e.g., about 10:1 or more).

A formulation comprising adjuvant and antigen or polynucleotide may be applied to skin of a human or animal subject, antigen is presented to immune cells, and an antigen-specific immune response is induced. This may occur before, during, or after infection by pathogen. Only antigen or polynucleotide encoding antigen may be required, but no additional adjuvant, if the immunogenicity of the formulation is sufficient to not require adjuvant activity. The formulation may include an additional antigen such that application of the formulation induces an immune response against multiple antigens (i.e., multivalent). In such a case, antigens may or may not be derived from the same source, but the antigens will have different chemical structures so as to induce immune responses specific for the different antigens. Antigen-specific lymphocytes may participate in the immune response and, in the case of participation by B lymphocytes, antigen-specific antibodies may be part of the immune response. The formulations described above may include binders, buffers, colorings, dessicants, diluents, humectants, preservatives, stabilizers, other excipients, adhesives, plasticizers, tackifiers, thickeners, and patch materials known in the art.

The invention is used to treat a subject (e.g., a human or animal in need of treatment such as prevention of disease, protection from effects of infection, therapy of existing disease or symptoms, or combinations thereof). Diseases other than infection include cancer, allergy, and autoimmunity. When the antigen is derived from a pathogen, the treatment may vaccinate the subject against infection by the pathogen or against its pathogenic effects such as those caused by toxin secretion. The invention may be used therapeutically to treat existing disease, protectively to prevent disease, to reduce the severity and/or duration of disease, to ameliorate symptoms of disease, or combinations thereof.

The application site may be protected with anti-inflammatory corticosteroids such as hydrocortisone, triamcinolone and mometazone or nonsteroidal anti-inflammatory drugs (NSAID) to reduce possible local skin reaction or modulate the type of immune response. Similarly, anti-inflammatory steroids or NSAID may be included in the patch material, or liquid or solid formulations; and corticosteroids or NSAID may be applied after immunization. IL-10, TNF-α, other immunomodulators may be used instead of the anti-inflammatory agents. Moreover, the formulation may be applied to skin overlying more than one draining lymph node field using either single or multiple applications. The formulation may include additional antigens such that application induces an immune response to multiple antigens. In such a case, the antigens may or may not be derived from the same source, but the antigens will have different chemical structures so as to induce an immune response specific for the different antigens. Multi-chambered patches could allow more effective delivery of multivalent vaccines as each chamber covers different antigen presenting cells. Thus, antigen presenting cells would encounter only one antigen (with or without adjuvant) and thus would eliminate antigenic competition and thereby enhancing the response to each individual antigen in the multivalent vaccine.

The formulation may be epicutaneously applied to skin to prime or boost the immune response in conjunction with or without penetration techniques, or other routes of immunization. Priming by transcutaneous immunization (TCI) with either single or multiple applications may be followed with enteral, mucosal, transdermal, and/or other parenteral techniques for boosting immunization with the same or altered antigens. Priming by an enteral, mucosal, transdermal, and/or other parenteral route with either single or multiple applications may be followed with transcutaneous techniques for boosting immunization with the same or altered antigens. It should be noted that TCI is distinguished from conventional topical techniques like mucosal or transdermal immunization because the former requires a mucous membrane (e.g., lung, mouth, nose, rectum) not found in the skin and the latter requires perforation of the skin through the dermis; The formulation may include additional antigens such that application to skin induces an immune response to multiple antigens.

In addition to antigen and adjuvant, the formulation may comprise a vehicle. For example, the formulation may comprise an AQUAPHOR, Freund, Ribi, or Syntex emulsion; water-in-oil emulsions (e.g., aqueous creams, ISA-720), oil-in-water emulsions (e.g., oily creams, ISA-51, MF59), microemulsions, anhydrous lipids and oil-in-water emulsions, other types of emulsions; gels, fats, waxes, oil, silicones, and humectants (e.g., glycerol).

Antigen may be derived from any pathogen that infects a human or animal subject (e.g., bacterium, virus, fungus, or protozoan), allergens, and self-antigens. The chemical structure of the antigen may be described as one or more of carbohydrate, fatty acid, and protein (e.g., glycolipid, glycoprotein, lipoprotein). Proteinaceous antigen is preferred. The molecular weight of the antigen may be greater than 500 daltons, 800 daltons, 1000 daltons, 10 kilodaltons, 100 kilodaltons, or 1000 kilodaltons (including intermediate ranges thereof). Chemical or physical penetration enhancement may be preferred for macromolecular structures like cells, viral particles, and molecules of greater than one megadalton, but techniques like hydration and swabbing with a solvent may be sufficient to induce immunization across the skin. Antigen may be obtained by recombinant techniques, chemical synthesis, or at least partial purification from a natural source. It may be a chemical or recombinant conjugates: for example, linkage between chemically reactive groups or protein fusion. Antigen may be provided as a live cell or virus, an attenuated live cell or virus, a killed cell, or an inactivated virus. Alternatively, antigen may be at least partially purified in cell-free form (e.g., cell or viral lysate, membrane or other subcellular fraction). Because most adjuvants would also have immunogenic activity and would be considered antigens, adjuvants would also be expected to have the aforementioned properties and characteristics of antigens. For example, adjuvants and antigens may be prepared using the same techniques (see above).

The choice of adjuvant may allow potentiation or modulation of the immune response. Moreover, selection of a suitable adjuvant may result in the preferential induction of a humoral or cellular immune response, specific antibody isotypes (e.g., IgM, IgD, IgA1, IgA2, IgE, IgG1, IgG2, IgG3, and/or IgG4), and/or specific T-cell subsets (e.g., CTL, Th1, Th2 and/or T_{DTH}). The adjuvant is preferably a chemically activated (e.g., proteolytically digested) or genetically activated (e.g., fusions, deletion or point mutants) ADP-ribosylating exotoxin or B subunit thereof.

An “antigen” is an active component of the formulation which is specifically recognized by the immune system of a human or animal subject after immunization or vaccination. The antigen may comprise a single or multiple immunogenic epitopes recognized by a B-cell receptor (i.e., secreted or membrane-bound antibody) or a T-cell receptor. Proteinaceous epitopes recognized by T-cell receptors have typical lengths and conserved amino acid residues depending on whether they are bound by major histocompatibility complex (MHC) Class I or Class II molecules on the antigen presenting cell. In contrast, proteinaceous epitopes recognized antibody may be of variable length including short, extended oligopeptides and longer, folded polypeptides. Single amino acid differences between epitopes may be distinguished. The antigen may be capable of inducing an immune response against a molecule of a pathogen, allergenic substances, or mammalian host (e.g., autoantigens, cancer antigens, molecules of the immune system). For immunoregulation, that molecule may be an allergen, autoantigen, internal image thereof, or other components of the immune system (e.g., B- or T-cell receptor, co-receptor or ligand thereof, soluble mediator or receptor thereof. Thus, antigen is usually identical or at least derived from the chemical structure of the molecule, but mimetics which are only distantly related to such chemical structures may also be successfully used.

An “adjuvant” is an active component of the formulation to assist in inducing an immune response to the antigen. Adjuvant activity is the ability to increase the immune response to a heterologous antigen (i.e., antigen which is a separate chemical structure from the adjuvant) by inclusion of the adjuvant itself in a formulation or in combination with other components of the formulation or particular immunization techniques. As noted above, a molecule may contain both antigen and adjuvant activities by chemically conjugating antigen and adjuvant or genetically fusing coding regions of antigen and adjuvant; thus, the formulation may contain only one ingredient or component. Some naturally-occurring proteins such as CT and LT have both adjuvant and antigenic properties; some recombinant proteins are known to have similar properties (LeIF); some non-protein adjuvants may also induce antibodies to themselves, such as LPS or lipid A. The combination of adjuvant and antigenic qualities may be used to induce protective immune responses. For example, LT antibodies are protective against ETEC, LeIF immune responses are effective in manifestations of Leishmaniasis and LPS antibodies may be protective in protection against diseases caused by gram-negative organisms.

The term “effective amount” is meant to describe that amount of adjuvant or antigen which induces an antigen-specific immune response. A “subunit” immunogen or vaccine is a formulation comprised of active components (e.g., adjuvant, antigen) which have been isolated from other cellular or viral components of the pathogen (e.g., membrane or polysaccharide components like endotoxin) by recombinant techniques, chemical synthesis, or at least partial purification from a natural source.

Induction of an immune response may provide treatments of a subject such as, for example, immunoprotection, desensitization, immunosuppression, modulation of autoimmune disease, potentiation of cancer immunosurveillance, prophylactic vaccination to prevent disease, and therapeutic vaccination to ameliorate established disease. A product or method “induces” when its presence or absence causes a statistically significant change in the immune response's magnitude and/or kinetics; change in the induced elements of the immune system (e.g., humoral vs. cellular, Th1 vs. Th2); effect on the number and/or the severity of disease symptoms; effect on the health and well-being of the subject (i.e., morbidity and mortality); or combinations thereof.

The term “draining lymph node field” as used in the invention means an anatomic area over which the lymph collected is filtered through a set of defined lymph nodes (e.g., cervical, axillary, inguinal, epitrochelear, popliteal, those of the abdomen and thorax). Thus, the same draining lymph node field may be targeted by immunization (e.g., enteral, mucosal, transcutaneous, transdermal, other parenteral,) within the few days required for antigen presenting cells to migrate to the lymph nodes if the sites and times of immunization are appropriately spaced to bring different components of the formulation together (e.g., two closely located patches with either adjuvant or antigen applied at the same time may be effective when neither alone would be successful). For example, a patch delivering adjuvant by the transcutaneous technique may be placed on the same arm as is injected with a conventional vaccine to boost its effectiveness in elderly, pediatric, or other immunologically compromised populations. In contrast, applying patches to different limbs may prevent an adjuvant-containing patch from boosting the effectiveness of a patch containing only antigen.

Without being bound to any particular theory for the operation of the invention but only to provide an explanation for our observations, we hypothesize that this transcutaneous delivery system carries antigen to cells of the immune system where an immune response is induced. The antigen may pass through the normally present protective outer layers of the skin (i.e., stratum corneum) and induce the immune response directly, or through an antigen presenting cell population in the epidermis (e.g., macrophage, tissue macrophage, Langerhans cell, other dendritic cells, B lymphocyte, or Kupffer cell) that presents processed antigen to lymphocytes. Thus, with or without penetration enhancement techniques, the dermis is not penetrated for TCI as it is for subcutaneous injection or transdermal techniques. Optionally, the antigen may pass through the stratum corneum via a hair follicle or a skin organelle (e.g., sweat gland, oil gland).

Transcutaneous immunization with bacterial ADP-ribosylating exotoxins (bARE) as an example, may target the epidermal Langerhans cell, known to be among the most efficient of the antigen presenting cells (APC). Maturation of APC may be assessed by morphology and phenotype (e.g., expression of MHC Class II molecules, CD83, or co-stimulatory molecules). We have found that bARE appear to activate Langerhans cells when applied epicutaneously to intact skin. Adjuvants such as trypsin-cleaved bARE may enhance Langerhans cell activation. Langerhans cells direct specific immune responses through phagocytosis of the antigens, and migration to the lymph nodes where they act as APC to present the antigen to lymphocytes, and thereby induce a potent antibody response. Although the skin is generally considered a barrier to pathogens, the imperfection of this barrier is attested to by the numerous Langerhans cells distributed throughout the epidermis that are designed to orchestrate the immune response against organisms invading through the skin. According to Udey (Clin. Exp. Immunol., 107:s6-s8, 1997):

Langerhans cells are bone-marrow derived cells that are present in all mammalian stratified squamous epithelia. They comprise all of the accessory cell activity that is present in uninflamed epidermis, and in the current paradigm are essential for the initiation and propagation of immune responses directed against epicutaneously applied antigens. Langerhans cells are members of a family of potent accessory cells (‘dendritic cells’) that are widely distributed, but infrequently represented, in epithelia and solid organs as well as in lymphoid tissue.

It is now recognized that Langerhans cells (and presumably other dendritic cells) have a life cycle with at least two distinct stages. Langerhans cells that are located in epidermis constitute a regular network of antigen-trapping ‘sentinel’ cells. Epidermal Langerhans cells can ingest particulates, including microorganisms, and are efficient processors of complex antigens. However, they express only low levels of MHC class I and II antigens and costimulatory molecules (ICAM-1, B7-1 and B7-2) and are poor stimulators of unprimed T cells. After contact with antigen, some Langerhans cells become activated, exit the epidermis and migrate to T-cell-dependent regions of regional lymph nodes where they localize as mature dendritic cells. In the course of exiting the epidermis and migrating to lymph nodes, antigen-bearing epidermal Langerhans cells (now the ‘messengers’) exhibit-dramatic changes in morphology, surface phenotype and function. In contrast to epidermal Langerhans cells, lymphoid dendritic cells are essentially non-phagocytic and process protein antigens inefficiently, but express high levels of MHC class I and class II antigens and various costimulatory molecules and are the most potent stimulators of naive T cells that have been identified.”

The potent antigen presenting capability of Langerhans cells can be exploited for transcutaneously-delivered immunogens and vaccines. An immune response using the skin's immune system may be achieved by delivering the formulation only to Langerhans cells in the stratum corneum (i.e., the outermost layer of the skin consisting of cornified cells and lipids) and subsequently activating the Langerhans cells to take up antigen, migrate to B-cell follicles and/or T-cell dependent regions, and present the antigen to B and/or T lymphocytes. If antigens other that bARE (e.g., toxin, colonization or virulence factor) are to be phagocytosed by Langerhans cells, then these antigens could also be transported to the lymph node for presentation to T lymphocytes and subsequently induce an immune response specific for that antigen. Thus, a feature of TCI is the activation of the Langerhans cell, presumably by bARE or derivatives thereof, chemokines, cytokines, PAMP, or other Langerhans cell activating substance including contact sensitizers and adjuvants. Increasing the size of the skin population of Langerhans cells or their state of activation would also be expected to enhance the immune response (e.g., acetone pretreatment). In aged subjects or Langerhans cell-depleted skin (i.e., from UV damage), it may be possible to replenish the population of Langerhans cells (e.g., tretinoin pretreatment).

Adjuvants such as bARE are known to be highly toxic when injected or given systemically. Intradermal injection has also been shown to induce persistent nodules when LT is included as the adjuvant (Guy et al., Vaccine, 17:1130-1135, 1999). But if placed on the surface of intact skin (ie., epicutaneous), they are unlikely to induce systemic toxicity. Thus, the transcutaneous route may allow the advantage of adjuvant effects without systemic toxicity. A similar absence of toxicity could be expected if the skin were penetrated only below the stratum corneum (e.g., near or at the epidermis), but not through the dermis. Thus, the ability to induce activation of the immune system through the skin induces potent immune responses without systemic toxicity.

The magnitude of the antibody response induced by affinity maturation and isotype switching to predominantly IgG antibodies is generally achieved with T-cell help, and activation of both Th1 and Th2 pathways is suggested by the production of IgG1 and IgG2a. Alternatively, a large antibody response may be induced by a thymus-independent antigen type 1 (TI-1) which directly activates the B lymphocyte or could have similar activating effects on B lymphocytes such as up-regulation of MHC Class II, CD25, CD40, B7-1/CD80, B7-2/CD86, and ICAM-1 molecules.

The spectrum of commonly known skin immune responses is represented by atopy and contact dermatitis. Contact dermatitis, a pathogenic manifestation of Langerhans cell activation, is directed by Langerhans cells which phagocytose antigen, migrate to lymph nodes, present antigen, and sensitize T lymphocytes that migrate to the skin and cause the intense destructive cellular response that occurs at affected skin sites. Such responses are not generally known to be associated with antigen-specific IgG antibodies. Atopic dermatitis may utilize the Langerhans cell in a similar fashion, but is identified with Th2 cells and is generally associated with high levels of IgE antibody.

On the other hand, transcutaneous immunization with bARE provides a useful and desirable immune response. There are usually no findings typical of atopy or contact dermatitis given the high levels of IgG that are induced. Cholera toxin or E. coli heat-labile enterotoxin epicutaneously applied to skin can achieve immunization in the absence of lymphocyte infiltration 24, 48 and 120 hours after immunization. The minor skin reactivity seen in preclinical and clinical trials were easily treated. This indicates that Langerhans cells engaged by transcutaneous immunization as they “comprise all of the accessory cell activity that is present in uninflamed epidermis, and in the current paradigm are essential for the initiation and propagation of immune responses directed against epicutaneously applied antigens” (Udey, 1997). The uniqueness of the transcutaneous immune response here is also indicated by both the high levels of antigen-specific IgG antibody and the type of antibody produced (e.g., IgM, IgG1, IgG2a, IgG2b, IgG3 and IgA), and generally the absence of antigen specific IgE antibody. Transcutaneous immunization could conceivably occur in tandem with skin inflammation if sufficient activation of antigen presenting cells and T lymphocytes were to occur in a transcutaneous response coexisting with atopy or contact dermatitis.

Transcutaneous targeting of Langerhans cells may also be used in tandem with agents to deactivate all or part of their antigen presenting function, thereby modifying immunization or preventing sensitization. Techniques to modulate Langerhans activation or other skin immune cells include, for example, the use of anti-inflammatory steroidal or nonsteroidal agents (NSAID); cyclosporin, FK506, rapamycin, cyclophosphamide, glucocorticoids, or other immunosuppressants; interleukin-10; interleukin-1 monoclonal antibodies (mAB) or soluble receptor antagonists (RA); interleukin-1 converting enzyme (ICE) inhibitors; or depletion via superantigens such as through Staphylococcal enterotoxin A (SEA) induced epidermal Langerhans cell depletion. Similar compounds may be used to modify the innate response of Langerhans cells and induce different T-helper responses (Th1 or Th2) or may modulate skin inflammatory responses to decrease potential side effects of the immunization. Similarly, lymphocytes may be immunosuppressed before, during or after immunization by administering immunosuppressant separately or by coadministration of immunosuppressant with the formulation. For example, it may be possible to induce a potent systemic protective immune responses with agents that would normally result in allergic or irritant contact hypersensitivity but adding inhibitors of ICE may alleviate adverse skin reactions.

Antigen

A transcutaneous immunization system delivers agents to specialized cells (e.g., antigen presentation cell, lymphocyte) that produce an immune response. These agents as a class are called antigens. Antigen may be composed of chemical structures such as, for example, carbohydrate, glycolipid, glycoprotein, lipid, lipoprotein, phospholipid, polypeptide, conjugates thereof, or any other material known to induce an immune response. Antigen may be conjugated to carrier. Antigen may be provided as a whole organism such as, for example, a bacterium or virion; antigen may be obtained from an extract or lysate, either from whole cells or membrane alone; or antigen may be chemically synthesized or produced by recombinant technology. Antigen may be incorporated into a formulation by solubilization or dispersion.

Antigen of the invention may be expressed by recombinant technology, preferably as a fusion with an affinity or epitope tag; chemical synthesis of an oligopeptide, either free or conjugated to carrier proteins, may be used to obtain antigen of the invention. Oligopeptides are considered a type of polypeptide. Oligopeptide lengths of 6 residues to 20 residues are preferred. Polypeptides may also by synthesized as branched structures. Antigenic polypeptides include, for example, synthetic or recombinant B-cell and T-cell epitopes, universal T-cell epitopes, and mixed T-cell epitopes from one organism or disease and B-cell epitopes from another. Antigen obtained through recombinant technology or peptide synthesis, as well as antigen obtained from natural sources or extracts, may be purified by the antigen's physical and chemical characteristics, preferably by fractionation or chromatography. Recombinants may combine antigen fragments or fuse them into chimerae. A multivalent antigen formulation may be used to induce an immune response to more than one antigen at the same time. Conjugates may be used to induce an immune response to multiple antigens, to boost the immune response, or both. Transcutaneous immunization may be used to boost responses induced initially by other routes of immunization such as by oral, nasal or other parenteral routes. Such oral/transcutaneous or transcutaneous/oral immunization may be especially important to enhance mucosal immunity in diseases where mucosal immunity correlates with protection.

Antigen may be solubilized in a buffer or water or organic solvents such as alcohol or DMSO, or incorporated in gels, emulsions, lipid micelles or vesicles, and creams. Suitable buffers include, but are not limited to, phosphate buffered saline Ca⁺⁺/Mg⁺⁺ free, phosphate buffered saline, normal saline (150 mM NaCl in water), and Hepes or Tris buffer. Antigen not soluble in neutral buffer can be solubilized in 10 mM acetic acid and then diluted to the desired volume with a neutral buffer such as PBS. In the case of antigen soluble only at acid pH, acetate-PBS at acid pH may be used as a diluent after solubilization in dilute acetic acid. Dimethyl sulfoxide and glycerol may be suitable nonaqueous buffers for use in the invention.

A hydrophobic antigen can be solubilized in a detergent or surfactant, for example a polypeptide containing a membrane-spanning domain. Furthermore, for formulations containing liposomes, an antigen in a detergent solution (e.g., cell membrane extract) may be mixed with lipids, and liposomes then may be formed by removal of the detergent by dilution, dialysis, or column chromatography. Certain antigens (e.g., membrane proteins) need not be soluble per se, but can be inserted directly into a lipid membrane (e.g., virosome), in a suspension of virion alone, or suspensions of microspheres or heat-inactivated bacteria which may be taken up by activate antigen presenting cells (e.g., opsonization). Antigens may also be mixed with a penetration enhancer as described in WO 99/43350.

Many antigens are known in the art which can be used to vaccinate human or animal subjects and induce an immune response specific for particular pathogens, as well as methods of preparing antigen, determining a suitable dose of antigen, assaying for induction of an immune response, and treating infection by a pathogen (e.g., bacterium, virus, fungus, or protozoan). Environmental and food allergens, as well as self-antigens of the mammalian host (e.g., human, animal) are examples of antigens that are not derived from a pathogen. Antigen used to produce formulations and vaccines for transcutaneous immunization may be the natural product per se, genetically-engineered or chemically-synthesized forms thereof, fragments thereof, fusions, or conjugates. The immune response will usually recognize only a portion of the antigen (e.g., one or more immunogenic epitopes).

Plotkin and Mortimer (Vaccine, 2^{nd} Ed., Philadelphia: W. B. Saunders, 1994) provide antigens which can be used to vaccinate humans or animals to induce an immune response specific for particular pathogens, as well as methods of preparing antigen, determining a suitable dose of antigen, assaying for induction of an immune response, and treating infection by a pathogen.

Bacteria include, for example: anthrax, Campylobacter, Viblio cholera, clostridia including Clostridium difficile, Diphtheria, enterohemorrhagic E. coli, enterotoxigenic E. coli, Giardia, gonococcus, Helicobacter pylori, Hemophilus influenza B, Hemophilus influenza nontypeable, Legionella, meningococcus, Mycobacteria including those organisms responsible for tuberculosis, pertussis, pneumococcus, salmonella, shigella, staphylococcus, Group A beta-hemolytic streptococcus, Streptococcus B, tetanus, Borrelia burgdorfi and Yersinia. Products thereof which may be used as antigen. Antigen includes, for example, toxins, toxoids, subunits thereof, or combinations thereof; virulence or colonization factors; and products.

Viruses include, for example:adenovirus, dengue serotypes 1 to 4, ebola, enterovirus, hanta virus, hepatitis serotypes A to E, herpes simplex virus 1 or 2, human immunodeficiency virus, human papilloma virus, influenza, measles, Norwalk, Japanese equine encephalitis, papilloma virus, parvovirus B19, polio, rabies, respiratory syncytial virus, rotavirus, rubella, rubeola, St. Louis encephalitis, vaccinia, viral expression vectors containing genes coding for other antigens such as malaria antigens, varicella, and yellow fever. The viral products or derivatives thereof may be used as sources for antigen.

Fungi including entities responsible for tinea corporis, tinea unguis, sporotrichosis, aspergillosis, candida and other pathogenic fungi. The fungal products or derivatives thereof may be used as sources for antigen.

Protozoans include, for example: Entamoeba histolytica, Plasmodium, Leishmania, and the Helminthes; Schistosomes; and products thereof. The protozoan products or derivatives thereof may be used as sources for antigen.

Of particular interest are pathogens that enter on or through mucosal surfaces such as, for example, pathogenic species in the bacterial genera Actinomyces, Aeromonas, Bacillus, Bacteroides, Bordetella, Brucella, Campylobacter, Capnocytophaga, Clamydia, Clostridium, Corynebacterium, Eikenella, Erysipelothnx, Escherichia, Fusobacterium, Hemophilus, Klebsiella, Legionella, Leptospira, Listena, Mycobacterium, Mycoplasma, Neisseria, Nocardia, Pasteurella, Proteus, Pseudomonas, Rickettsia, Salmonella, Selenomonas, Shigella, Staphylococcus, Streptococcus, Treponema, Vibrio, and Versinia; pathogenic viral strains from the groups Adenovirus, Coronavirus, Herpesvirus, Orthomyxovirus, Picomovirus, Poxvirus, Reovirus, Retrovirus, Rotavirus; pathogenic fungi from the genera Aspergillus, Blastomyces, Candida, Coccidiodes, Cryptococcus, Histoplasma, and Phycomyces; and pathogenic protozoans in the genera Eimeria, Entamoeba, Giardia, and Trichomonas.

Vaccination has also been used as a treatment for cancer, allergies, and autoimmune disease. For example, vaccination with tumor antigen (e.g., HER2, prostate specific antigen) may induce an immune response in the form of antibodies, CTLs and lymphocyte proliferation which allows the body's immune system to recognize and kill tumor cells. Tumor antigens useful for vaccination have been described for leukemia, lymphoma, and melanoma. Allergens are known for animals (e.g., bird, cat, dog, rodents), cockroaches, fleas, mites, and plant pollen (e.g., grasses, trees). Vaccination with T-cell receptor or autoantigens (e.g., pancreatic islet antigen) may induce an immune response that halts progression of autoimmune disease.

Adjuvant

The formulation contains an adjuvant, although a single molecule may contain both adjuvant and antigen properties (e.g., ADP-ribosylating exotoxin). Because most adjuvants would also have immunogenic activity and would be considered antigens, adjuvants would also be expected to have the aforementioned properties and characteristics of antigens. For example, adjuvants and antigens may be prepared using the same techniques (see above).

Adjuvants are substances that are used to specifically or nonspecifically potentiate an antigen-specific immune response, perhaps through activation of antigen presenting cells (e.g., dendritic cells in various layers of the skin, especially Langerhans cells). See also Elson et al. (in Handbook of Mucosal Immunology, Academic Press, 1994). Although activation may initially occur in the epidermis or dermis, the effects may persist as the dendritic cells migrate through the lymph system and the circulation. Adjuvant may be formulated and applied with or without antigen, but generally, activation of antigen presenting cells by adjuvant occurs prior to presentation of antigen. Alternatively, they may be separately presented within a short interval of time but targeting the same anatomical region (e.g., the same draining lymph node field).

Adjuvants include, for example, chemokines (e.g., defensins, HCC-1, HCC-4, MCP-1, MCP-3, MCP-4, MIP-1α, MIP-1β, MIP-1δ, MIP-3α, MIP-2, RANTES); other ligands of chemokine receptors (e.g., CCR1, CCR-2, CCR-5, CCR6, CXCR-1); cytokines (e.g., IL-1β, IL-2, IL6, IL8, IL-10, IL-12; IFN-γ; TNF-α; GM-CSF); other protein ligands of receptors for those cytokines, heat shock proteins and derivatives thereof; Leishmania homologs of elF4a and derivatives thereof; bacterial ADP-ribosylating exotoxins and derivatives thereof (e.g., genetic mutants, A and/or B subunit-containing fragments, chemically toxoided versions); chemical conjugates or genetic recombinants containing bacterial ADP-ribosylating exotoxins or derivatives thereof; C3d tandem array; and superantigens. See also Nohria et al. (Biotherapy, 7:261-269, 1994) and Richards et al. (in Vaccine Design, Eds. Powell et al., Plenum Press, 1995) for other useful adjuvants.

Adjuvant may be chosen to preferentially induce antibody or cellular effectors, specific antibody isotypes (e.g., IgM, IgD, IgA1, IgA2, secretory IgA, IgE, IgG1, IgG2, IgG3, and/or IgG4), or specific T-cell subsets (e.g., CTL, Th1, Th2 and/or T_{DTH}). For example, antigen presenting cells may present Class II-restricted antigen to precursor CD4+T cells, and the Th1 or Th2 pathway may be entered. T helper cells actively secreting cytokine are primary effector cells; they are memory cells if they are resting. Reactivation of memory cells produces memory effector cells. Th1 characteristically secrete IFN-γ (TNF-β and IL-2 may also be secreted) and are associated with “help” for cellular immunity, while Th2 characteristically secrete IL-4 (IL-5 and IL-13 may also be secreted) and are associated with “help” for humoral immunity. Depending on disease pathology, adjuvants may be chosen to prefer a Th1 response (e.g., antigen-specific cytolytic cells) vs. a Th2 response (e.g., antigen specific antibodies).

Most ADP-ribosylating exotoxins (bARE) are organized as A:B heterodimers with a B subunit containing the receptor binding activity and an A subunit containing the ADP-ribosyltransferase activity. Exemplary bARE include cholera toxin (CT) E. coli heat-labile enterotoxin (LT), diphtheria toxin, Pseudomonas exotoxin A (ETA), pertussis toxin (PT), C. botulinum toxin C2, C. botulinum toxin C3, C. limosum exoenzyme, B. cereus exoenzyme, Pseudomonas exotoxin S, S. aureus EDIN, and B. sphaericus toxin. Mutant bARE, for example containing mutations of the trypsin cleavage site (e.g., Dickenson et al., Infect Immun, 63:1617-1623, 1995) or mutations affecting ADP-ribosylation (e.g., Douce et al., Infect Immun, 65:28221-282218,1997) may be used.

TCI may be accompished through the ganglioside GM₁ binding activity of CT, LT, or subunits thereof (e.g., CTB or LTB). Ganglioside GM₁ is a ubiquitous cell membrane glycolipid found in all mammalian cells. When the pentameric CT B subunit binds to the cell surface, a hydrophilic pore is formed which allows the A subunit to insert across the lipid bilayer. Other binding targets on the APC may be utilized (e.g., ETA binds α₂ macroglobulin receptor-low density lipoprotein receptor-related protein). The LT B subunit binds to ganglioside GM, in addition, to other gangliosides and its binding activities may account for its the fact that LT is highly immunogenic on the skin.

TCI with bARE or B subunit-containing fragments or conjugates thereof may require their ganglioside GM₁ binding activity. When mice were transcutaneously immunized with CT, CTA and CTB, CT and CTB were required for induction of an immune response. CTA contains the ADP-ribosylating exotoxin activity but only CT and CTB containing the binding activity are able to induce an immune response indicating that the B subunit was necessary and sufficient to immunize through the skin. We conclude that the Langerhans cells or other APC may be activated by CTB binding to its cell surface resulting in a transcutaneous immune response.

CT, LT, ETA and PT, despite having different cellular binding sites, are potent adjuvants for transcutaneous immunization, inducing IgG antibodies but not IgE antibodies. CTB without CT can also induce IgG antibodies. Thus, both bARE and a derivative thereof can effectively immunize when epicutaneously applied to the skin. Native LT as an adjuvant and antigen, however, is clearly not as potent as native CT. But activated bARE can act as adjuvants for weakly immunogenic antigens in a transcutaneous immunization system. Thus, therapeutic immunization with one or more antigens could be used separately or in conjunction with immunostimulation of the antigen presenting cell to induce a prophylactic or therapeutic immune response.

In general, toxins can be chemically inactivated to form toxoids which are less toxic but remain immunogenic. We envision that the transcutaneous immunization system using toxin-based immunogens and adjuvants can achieve anti-toxin levels adequate for protection against these diseases. The anti-toxin antibodies may be induced through immunization with the toxins, or genetically-detoxified toxoids themselves, or with toxoids and adjuvants. Genetically toxoided toxins which have altered ADP-ribosylating exotoxin activity or trypsin cleavage site, but not binding activity, are envisioned to be especially useful as nontoxic activators of antigen presenting cells used in transcutaneous immunization and may reduce concerns over toxin use.

bARE can also act as an adjuvant to induce antigen-specific CTL through transcutaneous immunization. The bARE adjuvant may be chemically conjugated to other antigens including, for example, carbohydrates, polypeptides, glycolipids, and glycoprotein antigens. Chemical conjugation with toxins, their subunits, or toxoids with these antigens would be expected to enhance the immune response to these antigens when applied epicutaneously. To overcome the problem of the toxicity of the toxins (e.g., diphtheria toxin is known to be so toxic that one molecule can kill a cell) and to overcome the problems of working with such potent toxins as tetanus, several workers have taken a recombinant approach to producing genetically-produced toxoids. This is based on inactivating the catalytic activity of the ADP-ribosyl transferase by genetic deletion. These toxins retain the binding capabilities, but lack the toxicity, of the natural toxins. Such genetically toxoided exotoxins would be expected to induce a transcutaneous immune response and to act as adjuvants. They may provide an advantage in a transcutaneous immunization system in that they would not create a safety concern as the toxoids would not be considered toxic. Activation through a technique such as trypsin cleavage, however, would be expected to enhance the adjuvant qualities of LT through the skin which lacks trypsin-like enzymes. Additionally, several techniques exist to chemically modify toxins and can address the same problem. These techniques could be important for certain applications, especially pediatric applications, in which ingested toxins might possibly elicit adverse reactions.

Adjuvant may be biochemically purified from a natural source (e.g., pCT or pLT) or recombinantly produced (e.g., rCT or rLT). ADP-ribosylating exotoxin may be purified either before or after proteolysis (i.e., activation). B subunit of the ADP-ribosylating exotoxin may also be used: purified from the native enzynie after proteolysis or produced from a fragment of the entire coding region of the enzyme. The subunit of the ADP-ribosylating exotoxin may be used separately (e.g., CTB or LTB) or together (e.g., CTA-LTB, LTA-CTB) by chemical conjugation or genetic fusion. A fragment of the ADP-ribosylating exotoxin which retains the ability to bind its cell membrane receptor may also be biochemically purified or recombinantly produced, and then used instead of the B subunit.

Point mutations (e.g., single, double, or triple amino acid substitutions), deletions (e.g., protease recognition site), and isolated functional domains of ADP-ribosylating exotoxin may also be used as adjuvant. Derivatives which are less toxic or have lost their ADP-ribosylation activity, but retain their adjuvant activity have been described. Specific mutants of E. coli heat-labile enterotoxin include LT-K63, LT-R72, LT(H44A), LT(R192G), LT(R192G/1211A), and LT(Δ192-194). Toxicity may be assayed with the Y-1 adrenal cell assay (Clements and Finkelstein, Infect. Immun., 24:760-769, 1979). ADP-ribosylation may be assayed with the NAD-agmatine ADP-ribosyltransferase assay (Moss et al., J. Biol. Chem., 268:6383-6387, 1993). Particular ADP-ribosylating exotoxins, derivatives thereof, and processes for their production and characterization are described in U.S. Pat. Nos. 4,666,837; 4,935,364; 5,308,835; 5,785,971; 6,019,982; 6,033,673; and 6,149,919.

An activator of Langerhans cells may also be used as an adjuvant. Examples of such activators include proteins like chemokines, cytokines, differentiation factors, and growth factors (e.g., members of the TGFβ superfamily).

If an immunizing antigen has sufficient Langerhans cell activating capabilities then a separate adjuvant may not be required, as in the case of LT which is both antigen and adjuvant. Alternatively, such antigens can be considered not to require an adjuvant because they are sufficiently immunogenic. It may also be possible to use low concentrations of activators of Langerhans cells to induce an immune response without inducing skin lesions.

Other techniques for enhancing activity of adjuvants may be effective, such as adding surfactants and/or phospholipids to the formulation to enhance adjuvant activity of ADP-ribosylating exotoxin by ADP-ribosylation factor. One or more ADP-ribosylation factors (ARF) may be used to enhance the adjuvanticity of bARE (e.g., ARF1, ARF2, ARF3, ARF4, ARF5, ARF6, ARDL). Similarly, one or more ARF could be used with an ADP-ribosylating exotoxin to enhance its adjuvant activity.

Undesirable properties or harmful side effects (e.g., allergic or hypersensitive reaction; atopy, contact dermatitis, or eczema; systemic toxicity) may be reduced by modification without destroying its effectiveness in transcutaneous immunization. Modification may involve, for example, removal of a reversible chemical modification (e.g., proteolysis) or encapsulation in a coating which reversibly isolates one or more components of the formulation from the immune system. For example, one or more components of the formulation may be encapsulated in a particle for delivery (e.g., microspheres, nanoparticles) although we have shown that encapsulation in lipid vesicles is not required for transcutaneous immunization and appears to have a negative effect. Phagocytosis of a particle may, by itself, enhance activation of an antigen presenting cell by upregulating expression of MHC Class I and/or Class II molecules and/or costimulatory molecules (e.g., CD40, B7 family members like CD80 and CD86). Alternative methods of upregulating such molecules by activating an antigen presenting cell are also known (see above).

Formulation

Processes for manufacturing a pharmaceutical formulation are well known. The components of the formulation may be combined with a pharmaceutically-acceptable carrier or vehicle, as well as any combination of optional additives (e.g., at least one binder, buffer, coloring, dessicant, diluent, humectant, preservative, stabilizer, other excipient, or combinations thereof). See, generally, Ullmann's Encyclopedia of Industrial Chemistry, 6^{th} Ed. (electronic edition, 1998); Remington's Pharmaceutical Sciences, 22^{nd} (Gennaro, 1990, Mack Publishing); Pharmaceutical Dosage Forms, 2^{nd} Ed. (various editors, 1989-1998, Marcel Dekker); and Pharmaceutical Dosage Forms and Drug Delivery Systems (Ansel et al., 1994, Williams & Wilkins).

Good manufacturing practices are known in the pharmaceutical industry and regulated by government agencies (e.g., Food and Drug Administration). A liquid formulation may be prepared by dissolving an intended component of the formulation in a sufficient amount of an appropriate solvent. Generally, dispersions are prepared by incorporating the various components of the formulation into a vehicle which contains the dispersion medium. For production of a solid form from a liquid formulation, solvent may be evaporated at room temperature or in an oven. Blowing a stream of nitrogen or air over the surface accelerates drying; alternatively, vacuum drying or freeze drying can be used.

Suitable procedures for making the various dosage forms and production of patches are known. The size of each dose and the interval of dosing to the subject may be used to determine a suitable size and shape of the container, compartment, or chamber. Formulations will contain an effective amount of the active ingredients (e.g., at least one adjuvant and/or one or more antigens) together with carrier or suitable amounts of vehicle in order to provide pharmaceutically-acceptable compositions suitable for administration to a human or animal. Formulation that include a vehicle may be in the form of a cream, emulsion, gel, lotion, ointment, paste, solution, suspension, or other liquid forms known in the art; especially those that enhance skin hydration. For a patch, successive coatings of formulation may be applied to the substrate or several formulation-containing layers may be laminated to increase its capacity for active ingredients.

The relative amounts of active ingredients within a dose and the dosing schedule may be adjusted appropriately for efficacious administration to a subject (e.g., animal or human). This adjustment may depend on the subject's particular disease or condition, and whether therapy or prophylaxis is intended. To simplify administration of the formulation to the subject, each unit dose would contain the active ingredients in predetermined amounts for a single round of immunization.

There are numerous causes of protein instability or degradation, including hydrolysis and denaturation. In the case of denaturation, the protein's conformation is disturbed and the protein may unfold from its usual globular structure. Rather than refolding to its natural conformation, hydrophobic interaction may cause clumping of molecules together (i.e., aggregation) or refolding to an unnatural conformation. Either of these results may entail diminution or loss of antigenic or adjuvant activity. Stabilizers may be added to lessen or prevent such problems.

The formulation, or any intermediate in its production, may be pretreated with protective agents (i.e., cryoprotectants and drying stabilizers) and then subjected to cooling rates and final temperatures that minimize ice crystal formation. By proper selection of cryoprotective agents and the use of preselected drying parameters, almost any formulation might be dried for a suitable desired end use.

It should be understood in the following discussion of optional additives like binders, buffers, colorings, dessicants, diluents, humectants, preservatives, and stabilizers are described by their function. Thus, a particular chemical may act as some combination of the aforementioned. Such chemicals would be considered immunologically-inactive because they do not directly induce an immune response, but it increases the response by enhancing immunological activity of the antigen or adjuvant: for example, by reducing modification of the antigen or adjuvant, or denaturation during drying and hydrating cycles.

Stabilizers include dextrans and dextrins; glycols, alkylene glycols, polyalkane glycols, and polyalkylene glycols, sugars and starches, and derivatives thereof are suitable. Preferred additives are nonreducing sugars and polyols. In particular, trehalose, hydroxymethyl or hydroxyethyl cellulose, ethylene or propylene glycol, trimethyl glycol, vinyl pyrrolidone, and polymers thereof may be added. Alkali metal salts, ammonium sulfate, magnesium chloride, and surfactants (e.g., nonionic detergent), may stabilize proteinaceous adjuvants or antigens; optionally adding a carrier (e.g., agar, albumin, gelatin, glycogen, heparin), and freeze drying may further enhance stability. A polypeptide may also be stabilized by contacting it with a sugar such as, for example, a monosaccharide, disaccharide, sugar alcohol, and mixtures thereof (e.g., arabinose, fructose, galactose, glucose, lactose, maltose, mannitol, mannose, sorbitol, sucrose, xylitol). Polyols may stabilize a polypeptide, and are water-miscible or water-soluble. Various other excipients may also stabilize polpeptides, including amino acids, fatty acids and phospholipids, metals, reducing agents, and metal chelating agents. The stabilizer may be between 0.1% (w/v) and 10% (w/v) or between 1% (w/v) and 5% (w/v) of the adhesive formulation.

Single-dose formulations can be stabilized in poly(lactic acid) (PLA) and poly (lactide-co-glycolide) (PLGA) microspheres by suitable choice of stabilizer or other excipients. Trehalose may be advantageously used as an additive because it is a nonreducing saccharide, and therefore does not cause aminocarbonyl reactions with substances bearing amino groups such as proteins. Although stabilizers like high concentrations of sugar will combat the growth of microbes like bacteria and fungi, preservatives are typically antimicrobial agents that actively eliminate (e.g., bacteriocidal) or reduce the growth of microbes (e.g., bacteriostatic). Antioxidants may also be used to prevent oxidation of active ingredients of the formulation.

It is conceivable that a formulation or patch that can be administered to the subject in a dry, nonliquid (i.e., solid) form, may allow storage in conditions that do not require a cold chain. An antigen may be mixed with a heterologous adjuvant, placed on a dressing to form a patch, and allowed to completely dry. This dry patch can then be placed on skin with the dressing in direct contact with the skin for a period of time and be held in place covered with an occlusive backing layer (e.g., plastic or wax film).

Patch material may be nonwoven or woven (e.g., gauze dressing). Layers may also be laminated during processing. It may be nonocclusive or occlusive, but the latter is preferred for backing layers. The optional release liner preferably does not adsorb significant amounts of the formulation, perhaps by treating a film with silicone or fluorocarbon. The patch is preferably hermetically sealed for storage (e.g., foil packaging). The patch can be held onto the skin and components of the patch can be held together using various adhesives. One or more of the adjuvant and/or antigen may be applied to and/or incorporated in the adhesive portion of the patch. Generally, patches are planar and pliable, and they are manufactured with a uniform shape. Optional additives are plasticizers to maintain pliability of the patch, tackifiers to assist in adhesion between patch and skin, and thickeners to increase the viscosity of the formulation at least during processing.

Metal foil, cellulose, cloth (e.g., acetate, cotton, rayon), acrylic polymer, ethyl-enevinyl acetate copolymer, polyamide (e.g., nylon), polyester (e.g., poly-ethylene naphthalate, ethylene terephthalate), polyolefin (e.g., polyethylene, poly-propylene), polyurethane, polyvinylidene chloride (SARAN), natural or synthetic rubber, silicone elastomer, and combinations thereof are examples of patch materials (e.g., dressing, backing layer, release liner).

The adhesive may be an aqueous-based adhesive (e.g., acrylate or silicone). Acrylic adhesives are available from several commercial sources. Acrylic polymers may be a copolymer of C4-C18 aliphatic alcohol with methacrylic alkyl ester or the copolymer of methacrylic alkyl ester having C4-C18 alkyl, methacrylic acid, and/or other functional monomers. Examples of the methacrylic alkyl ester may include butyl acrylate, isobutyl acrylate, hexyl acrylate, octyl acrylate, 2-ethylhexyl acrylate, iso-octyl acrylate, decyl acrylate, isodecyl acrylate, lauryl acrylate, stearyl acrylate, methyl methacrylate, ethyl methacrylate, butyl methacrylate, isobutyl methacrylate, 2-ethylhexyl methacrylate, iso-octyl methacrylate, decyl methacrylate, etc.

Examples of the functional monomers may include a monomer containing hydroxyl group, a monomer containing carboxyl group, a monomer containing amide group, a monomer containing amino group. The monomer containing hydroxyl group may include hydroxyalkyl methacrylate such as 2-hydroxyethyl methacrylate, hydroxypropyl methacrylate and the like. The monomer containing carboxyl group may include α-β unsaturated carboxylic acid such as acrylic acid, methacrylic acid and the like; maleic mono alkyl ester such as butyl malate and the like; maleic acid; fumaric acid; crotonic acid and the like; and anhydrous maleic acid. Examples of the monomer containing amide group may include alkyl methacrylamide such as acryl-amide, dimethyl acrylamide, diethyl acrylamide and the like; alkylethylmethylol methacrylamide such as butoxymethyl acrylamide, ethoxymethyl acrylamide and the like; diacetone acrylamide; vinyl pyrrolidone; dimethyl aminoacrylate. In addition to the above exemplified monomers for copolymerization, vinyl acetate, styrene, α-methylstyrene, vinyl chloride, acrylonitrile, ethylene, propylene, butadiene and the like may be employed.

Commercially available acrylic adhesives are sold under the tradenames AROSET, DUROTAK, EUDRAGIT, GELVA, and NEOCRYL. EUDRAGIT polymers form a diverse family of polymers whose common feature is a polyacrylic or poly-methacrylic backbone that is compatible with the gastrointestinal tract and which have been widely used in pharmaceutical preparations, especially as coatings for tablets, but it has also been used as a coating for other medical devices. EUDRAGIT polymers are characterized as (1) an anionic copolymer based on methacrylic acid and methylmethacrylate wherein the ratio of free carboxyl groups to the ester groups is approximately 1:1, (2) an anionic copolymer based on methacrylic acid and methylmethacrylate wherein the ratio of free carboxyl groups to the ester groups is approximately 1:2, (3) a copolymer based on acrylic and methacrylic acid esters with a low content of quaternary ammonium groups wherein the molar ratio of the ammonium groups to the remaining neutral methacrylic acid esters is 1:20, and (4) a copolymer based on acrylic and methacrylic acid esters with a low content of quarternary ammonium groups wherein the molar ratio of the ammonium groups to the remaining neutral methacrylic acid esters is 1:40. The copolymers are sold under tradenames EUDRAGIT L, EUDRAGIT S, EUDRAGIT RL, and EUDRAGIT RS. EUDRAGIT E is a cationic copolymer based on diethylaminoethyl methacrylate and neutral methacrylic acid esters; EUDRAGIT NE is a neutral copolymer of polymethacrylates. For methacrylate or acrylate polymers, there are EUDRAGIT RS, EUDRAGIT RL, and EUDRAGIT NE; also available are EUDRAGIT RS-100, EUDRAGIT L-90, EUDRAGIT NE-30, EUDRAGIT L-100, EUDRAGIT S-100, EUDRAGIT E-100, EUDRAGIT RL-100, EUDRAGIT RS-100, EUDRAGIT RS-30D, EUDRAGIT E-100R, and EUDRAGIT RTM.

Furthermore, for the purpose of increasing or decreasing the water absorption capacity of an adhesive layer, the acrylic polymer may be copolymerized with hydrophilic monomer, monomer containing carboxyl group, monomer containing amide group, monomer containing amino group, and the like. Rubbery or silicone resins may be employed as the adhesive resin; they may be incorporated into the adhesive layer with a tackifying agent or other additives.

Alternatively, the water absorption capacity of the adhesive layer can be also regulated by incorporating therein highly water-absorptive polymers, polyols, and water-absorptive inorganic materials. Examples of the highly water-absorptive resins may include mucopolysaccharides such as hyaluronic acid, chondroitin sulfate, dermatan sulfate and the like; polymers having a large number of hydrophilic groups in the molecule such as chitin, chitin derivatives, starch and carboxy-methylcellulose; and highly water-absorptive polymers such as polyacrylic, polyoxyethylene, polyvinyl alcohol, and polyacrylonitrile. Examples of the water-absorptive inorganic materials, which may incorporated into the adhesive layer to regulate its water absorptive capacity, may include powdered silica, zeolite, powdered ceramics, and the like.

The plasticizer may be a trialkyl citrate such as, for example, acetyl-tributyl citrate (ATBC), acetyl-triethyl citrate (ATEC), and triethyl citrate (TEC). The plasticizer may be between 0.001% (w/v) and 5% (w/v) of the adhesive formulation. A suitable concentration may be empirically determined by selecting for pliability of the adhesive layer, and avoiding brittleness.

Exemplary tackifiers are glycols (e.g., glycerol, 1,3 butanediol, propylene glycol, polyethylene glycol); average molecular weights of 200, 300, 400, 800, 3000, etc. are available for the polyakylene glycols. Succinic acid is another tackifier. The tackifier may be between 0.1% (w/w) and 10% (w/w) of the adhesive formulation. A suitable concentration may be empirically determined by avoiding brittleness of the adhesive layer and its pliability.

Thickeners can be added to increase the viscosity of an adhesive or immunogenic formulation. The thickener may be a hydroxyalkyl cellulose or starch, or water-soluble polymers: for example, poloxamers, polyethylene oxides and derivatives thereof, polyethyleneimines, polyethylene glycols, and polyethylene glycol esters. But any molecule which serves to increase the viscosity of a solution may be suitable to improve handling of a formulation during manufacture of a patch. For example, hydroxyethyl or hydroxypropyl cellulose may be between 1% (w/w) and 10% (w/w) of the adhesive or immunogenic formulation. The formulation as a layer may be film cast or extruded, and then layers may be coated or laminated during manufacture of a patch. The capacity for protein might be increased by successive coatings or laminating several thin, adhesive layers together. Alternatively, a viscous formulation may be spread on a substrate (e.g., backing or adhesive layer) with minimal loss of immunologically-active ingredients like adjuvant or antigen. Thickeners are sold as NATROSOL hydroxyethyl cellulose and KLUCEL hydroxypropyl cellulose.

Gel and emulsion systems can be incorporated into patch delivery systems, or be manufactured separately from the patch, or added to the patch prior to application to the human or animal subject. Gels or emulsions may serve the same purpose of facilitating manufacture by providing a viscous formulation that can be easily manipulated with minimal loss. The term “gel” refers to covalently crosslinked, noncrosslinked hydrogel matrices. Hydrogels can be formulated with at least one protein with immunologic activity for PIA patches. Additional excipients may be added to the gel systems that allow for the enhancement of antigen/adjuvant delivery, skin hydration, and protein stability. The term “emulsion” refers to formulations such as water-in-oil creams, oil-in-water creams, ointments, and lotions. Emulsion systems can be either micelle-based, lipid vesicle-based, or both micelle- and lipid vesicle-based. Emulsion systems can be formulated with at least one adjuvant and/or antigen as the protein-in-adhesive systems. Additional excipients may be added to the emulsion systems that allow for the enhancement of antigen/adjuvant delivery, skin hydration, and protein stability.

Formulation may be applied with a patch in contact with skin of the subject. It may be covered with a nonocclusive or occlusive backing layer, the latter prevents evaporation and traps moisture at the site of application. Such a formulation may be applied to single or multiple sites, to single or multiple limbs, or to a large surface area of skin. Other substrates that may be used are pressure-sensitive adhesives such as acrylics, polyisobutylenes, and silicones. The formulation may be incorporated directly into such substrates, perhaps with the adhesive per se instead of adsorption to a porous pad (e.g., cotton gauze) or bilious strip (e.g., cellulose paper).

The adhesive and immunogenic formulations may be at least partially mixed or even throroughly blended, and then adhered to the backing layer. The immunologically-active ingredient may be dispersed or dissolved in the formulation. Alternatively the immunogenic formulation may be applied to the surface of the adhesive layer by coating or spreading over the adhesive using a Meyer rod, casting a layer and then laminating in close apposition with the adhesive u sing a roller, printing on the adhesive using a rotogravure, etc. Adhesive may be brought into contact with a release liner. Adhesive and immunogenic formulations may also be brought into contact with microblade or microneedle arrays or tines by coating, dipping the device into the formulation and drying, or spraying the device with the formulation.

Polymers added to the formulation may act as a stabilizer or other excipient of an active ingredient as well as reducing the concentration of the active ingredient that saturates a solution used to hydrate an at least partially-dried form (i.e., dry or semi-liquid) of the active ingredient. Such reduction occurs because the polymer reduces the effective free volume by filling “empty” space in the solvent. In this way, quantities of adjuvant/antigen can be conserved without reducing the amount of saturated solution. An important thermodynamic consideration is that an active ingredient in the saturated solution will be “driven” into regions of lower concentration (e.g., through the skin). For dispersal or dissolution of at least one adjuvant and/or one or more antigens, polymers can also stabilize the adjuvant/antigen-activity of those components of the formulation. Such polymers include ethylene or propylene glycol, vinyl pyrrolidone, and β-cyclodextrin polymers and copolymers.

Transcutaneous Delivery

Transcutaneous delivery of the formulation may target Langerhans cells and, thus, achieve effective and efficient immunization. Cells are found in abundance in the skin and are efficient antigen presenting cells (APC), which can lead to T-cell memory and potent immune responses. Because of the presence of large numbers of Langerhans cells in the skin, the efficiency of transcutaneous delivery may be related to the surface area exposed to antigen and adjuvant. In fact, the reason that transcutaneous immunization is so efficient may be that it targets a larger number of these efficient antigen presenting cells than intramuscular immunization.

Immunization may be achieved using epicutaneous application of a simple formulation of antigen and adjuvant, optionally covered by an occlusive dressing or using other patch technologies, to intact skin with or without chemical or physical penetration. Transcutaneous immunization according to the invention may provide a method whereby antigens and adjuvant can be delivered to the immune system, especially specialized antigen presentation cells underlying the skin (e.g., dendritic cells like Langerhans cells). The patch may be worn for as briefly as 30 sec; 1 min to 5 min; or less than 1 hour, 2 hours, 4 hours, 6 hours, 8 hours, 12 hours, 15 hours, 18 hours, 24 hours, or 48 hours. In contrast to transdermal patches delivering drugs, the release characteristics of the patch of the invention does not need to be constant or prolonged. It is preferred that the immunologically-active protein may be released quickly and quantitatively.

Moreover, transcutaneous immunization may be superior to immunization using hypodermic needles as more immune cells would be targeted by the use of several locations targeting large surface areas of skin. A therapeutically-effective amount of antigen sufficient to induce an immune response may be delivered transcutaneously either at a single cutaneous location, or over an area of skin covering multiple draining lymph node fields (e.g., cervical, axillary, inguinal, epitrochelear, popliteal, those of the abdomen and thorax). Such locations close to numerous different lymphatic nodes at locations all over the body will provide a more widespread stimulus to the immune system than when a small amount of antigen is injected at a single location by intradermal, subcutaneous, or intramuscular injection.

Antigen passing through or into the skin may encounter antigen presenting cells which process the antigen in a way that induces an immune response. Multiple immunization sites may recruit a greater number of antigen presenting cells and the larger population of antigen presenting cells that were recruited would result in greater induction of the immune response. It is conceivable that use of the skin may deliver antigen to phagocytic cells of the skin such as, for example, dendritic cells, Langerhans cells, macrophages, and other skin antigen presenting cells; antigen may also be delivered to phagocytic cells of the liver, spleen, and bone marrow that are known to serve as the antigen presenting cells through the blood stream or lymphatic system.

Langerhans cells, other dendritic cells, macrophages, or combinations thereof may be specifically targeted using their asialoglycoprotein receptor, mannose receptor, Fcγ receptor CD64, high-affinity receptor for IgE, or other highly expressed membrane proteins. A ligand or antibody specific for any of those receptors may be conjugated to or recombinantly produced as a protein fusion with adjuvant, antigen, or both. Furthermore, adjuvant, antigen, or both may be conjugated to or recombinantly produced as a protein fusion with protein A or protein G to target surface immunoglobulin of B lymphocytes. The envisioned result would be widespread distribution of antigen to antigen presenting cells to a degree that is rarely, if ever achieved, by current immunization practices.

A specific immune response may comprise humoral (i.e., antigen-specific antibody) and/or cellular (i.e., antigen-specific lymphocytes such as B lymphocytes, CD4⁺ T cells, CD8⁺ T cells, CTL, Th1cells, Th2 cells, and/or T_{DTH} cells) effector arms. Moreover, the immune response may comprise NK cells and other leukocytes that mediate antibody-dependent cell-mediated cytotoxicity (ADCC).

The immune response induced by the formulation of the invention may include the elicitation of antigen-specific antibodies and/or lymphocytes. Antibody can be detected by immunoassay techniques. Detection of the various antibody isotypes (e.g., IgM, IgD, IgA1, IgA2, secretory IgA, IgE, IgG1, IgG2, IgG3 or IgG4) can be indicative of a systemic or regional immune response. Immune responses can also be detected by a neutralizing assay. Antibodies are protective proteins produced by B lymphocytes. They are highly specific, generally targeting one epitope of an antigen. Immunization may induce antibodies that neutralize biological activity of an allergen, cell-entry receptor, growth factor receptor, or toxin. For example, inducing antibodies may treat a disease by specifically reacting with antigen (e.g., cholera toxin, HER2, influenza hemagluttinin) derived from a pathogen or cancer. Challenge studies in a host using infection by the pathogen or administration of toxin, comparison of morbidity or mortality between immunized and control populations, or measurement of another clinical criterion (e.g., high antibody titers or production of IgA antibody-secreting cells in mucosal membranes may be used as a surrogate marker) can demonstrate protection against disease or therapy of existing disease.

CTL are immune cells produced to protect against infection by a pathogen. They are also highly specific. Immunization may induce CTL specific for the antigen in association with self-major histocompatibility complex antigen. CTL induced by immunization with the transcutaneous delivery system may kill pathogen-infected cells or cancers. Immunization may also produce a memory response as indicated by boosting responses in antibodies and CTL, proliferation of lymphocyte cultures stimulated with the antigen, and delayed-type hypersensitivity (DTH) responses to intradermal skin challenge of the antigen alone.

The following is meant to be illustrative of the invention, but practice of the invention is not limited or restricted in any way by the following examples.

### EXAMPLES

Stability of Lysozyme-in-Adhesive Formulation

Many proteins and large biomolecules exhibit thermal lability, as well as chemical instability due to pH factors or incompatibility with a variety of compounds. Many adhesive systems are solvated in solvents which are detrimental to drug stability. Also many of these adhesive polymers contain functional groups which are incompatible with many reactive molecules. In addition, conventional technology often requires high temperatures to dry, extrude, or set the adhesive blend. It is therefore very difficult to formulate and process drug-in-adhesive systems for these compounds. The formulation and process described here allow for production of a protein-in-adhesive (PIA) system without thermal or chemical degradation of these delicate molecules. The formulation is also particularly suitable to large molecular weight biomolecules, owing to the water-soluble characteristics of the adhesive polymer which allow for release of the biomolecule when exposed to water.

Lysozyme was used as a model protein for the PIA formulation. Proteins can be chemically-labile compounds that aggregate, degrade, or otherwise denature when subjected to heat or a variety of solvents or reactive chemical sites. Lysozyme and assay of its enzymatic activity makes this a good model for proteins which needs stabilization.

Lysozyme was applied to patches made with two aqueous-based adhesives: lysozyme was applied to either a silicone adhesive or an acrylic adhesive on a patch. Protein was extracted from the adhesive in 20 ml water for 1 hr. Lysozyme recovery was about 85% to 90% from silicone-adhesive patches stored at room temperature or stored overnight at 40° C., as well as acrylic-adhesive patches stored at room temperature. Ground lysozyme was assayed as a positive control with a recovery of about 95% and activity of about 107% (bioactivity was assayed by UV spectroscopy and scan monitoring of reaction kinetics of lysozyme with Micrococcus lysodeikticus). Activity for the lysozyme extracted from adhesives of the patches was about 100% to 110% of original.

A methacrylate adhesive-KLUCEL thickener emulsion was prepared with a liquid plasticizer and emulsifier using water as the primary solvent at room temperature. Stability of the lysozyme in the wet blend was demonstrated for over seven days. The wet blend was coated soon after preparation and then dried using room temperature air or nitrogen, blown very close to the adhesive surface. The resulting partially-dried protein-in-adhesive demonstrated stability for over 30 days at room temperature as shown by lysozyme bioactivity. It also had acceptable adhesive and wear properties.

E coli Heat-Stable Enterotoxin (LT)-in-Adhesive Formulation

LT was obtained from Dr. John Clements of Tulane University (LTc). This material was obtained as a dry powder lyophilized from a TRIS buffer containing 200 mM NaCl. This LT has never been exposed to lactose. Unless otherwise noted, this is the source of LT used throughout this example. LT obtained from SSVI-Berne (LTs). This material was provided as a dry powder lyophilized from a phosphate buffered saline (PBS) formulation that contains 5% lactose. LTR192G or LT(R192G) mutant protein is a single amino acid residue mutant of LT: arginine at position 192 is mutated to glutamine. PBSₓ, pH 7.4 is 10 mM potassium phosphate buffered saline with pH of 7.4; the subscript x indicates the concentration of NaCl (e.g., PBS₂₀₀, pH7.4 has 200 mM NaCl).

KLUCEL EF thickener is hydroxypropyl cellulose, a viscosity enhancer made by Hercules, and was prepared as a 20% (w/w) stock in water. NATROSOL 250L NF is hydroxyethyl cellulose, a viscosity enhancer made by Hercules, and was prepared as a 12% (w/w) stock in water.

The adhesive fomulation is a suspension of EUDRAGIT EPO polymer (Rohm) in water containing 37.4% nonvolatile components (NVC). The modified adhesive formulation is the standard adhesive formulation to which was added 6% (w/w) glycerol and 4% (w/w) 1,3-butanediol. The final suspension contains 43.7% NVC. These additives are included to increase the plasticity and tackiness of cured films of EUDRAGIT adhesive.

Adhesive formulations have been identified in which LT shows good stability and recoverability. Wet blends contain about 500 μg/gm LT, 5% disaccharide (e.g., sucrose, trehalose), and 3% KLUCEL thickener. The blend is prepared by mixing EUDRAGIT adhesive and protein solution buffered at pH 7.4 (with disaccharide as nonreducing sugar and KLUCEL thickener) in a mass ratio of about 1:1. Excellent stability at 5° C. and good stability (there was some loss of recovered protein) at room temperature were observed over the course of 6 to 7 weeks.

Patches were manufactured by combining protein solutions (disaccharide containing) in a weight ratio of about 1:1 with standard EUDRAGIT adhesive. These wet blends were then cast as thin films on 1012 plastic backing using an 8-mil knife. The films were allowed to air dry at room temperature and then covered with a release liner. Patches with an area of about 1 cm² were punched out using a 7/16-inch diameter multi-purpose punch. Patches were placed in 5 ml glass lyophilization vials (with 20 mm mouth) and sealed under nitrogen. The sealed vials were placed on incubation and sampled at intervals.

Patches were rehydrated with ddH₂O, and the samples were prepared and analyzed according to the following procedures. EUDRAGIT adhesive is soluble under acidic conditions. This procedure can be used with ddH₂O, PBS₂₀, pH7.4, or SE-HPLC buffer (200 mM Na phosphate, pH7.2) as the rehydration buffer. Two patches without release liner were placed into a 1.7 ml EPPENDORF centrifuge tube. About 0.5 ml of rehydration buffer was added and the patches were able to rehydrate in buffer at room temperature for several hours with occasional manual agitation (about every half hour). The EPPENDORF tube was centrifuged for 5 min at 14,000 g and 4° C. The supernatant was recovered and used for HPLC analysis.

Reverse-phase high-performance liquid chromatography (RP-HPLC) was used to detect degradation of proteins such as LT and LTR192G. Protein subunits were eluted from a Vydac column (Protein & Peptide C4 with 2.1 mm ID×25 cm) at a rate of 0.3 ml/min using a gradient made from 0.1% (w/v) trifluoroacetic acid (TFA) in ddH₂O for buffer A and 0.1% (w/v) TFA in 95% (v/v) acetonitrile for buffer B. Both subunits A and B of LT were resolved, along with peaks for degraded protein and aggregated protein.

Denaturing polyacrylamide gel electrophoresis (SDS-PAGE) consisted of a 14% separating gel with a 5% stacking gel. Protein samples were reduced by boiling samples for 5 min in buffer containing β-mercaptoethanol. Separation of the A and B subunits with SDS-PAGE was used to confirm the RP-HPLC results.

Series 1A patches were prepared using LTs from SSVI Berne. Lyophilized samples were reconstituted with half the recommended volume of ddH₂O to give a solution with nominally 2 mg/ml of LT and 10% lactose in PBS. Trehalose was dissolved in this solution to a final concentration of 5% (w/v).

Series 2A patches were prepared using LTc from the Clements laboratory at Tulane University. LTc was reconstituted using ddH₂O and then dialyzed into PBS₁₅₀, pH7.4. The dialyzed LTc was concentrated using a 30,000 MW cutoff CENTRICON unit. Trehalose was dissolved in the concentrated LTc to give a final solution with 1.2 mg/ml LTc and 5% (w/v) trehalose in PBS₁₅₀, pH7.4.

Series 1A patches were incubated at 40° C. for one month. Series 2A patches were incubated at 40° C. for two months. LTc was better stabilized by a disaccharide: 5% (w/v) trehalose was a better stabilier for this protein than 10% (w/v) lactose. Peak heights in the chromatogram were lower and the ratio between the peak areas for A and B subunit products showed that there more degradation with lactose.

The standard EUDRAGIT adhesive was blended with other components (PBS buffer with LT, with or without trehalose, and with or without KLUCEL thickener) at a mass ratio of about 1:1.2 for blends with KLUCEL thickener, and at a mass ratio of about 1:1 for blends without KLUCEL thickener. Final concentrations in the wet blend were 3% (w/w) KLUCEL thickener, 5% (w/v) disaccharide (when present), and 410 μg/gm to 460 μg/gm LT.

Comparison of formulations containing trehalose to those without trehalose clearly shows that the presence of trehalose dramatically increased the stability of LT even at elevated temperatures as high as 60° C. Even in the absence of trehalose, patches prepared using standard EUDRAGIT adhesive showed greater LT stability than those prepared using modified EUDRAGIT adhesive. The purpose for modifying the EUDRAGIT adhesive, is to increase the tackiness and malleability of partially-dried films. Films of standard EUDRAGIT adhesive that included trehalose had a tendency to crumble and flake off the patch backing substrate. However, this flakiness dramatically decreased (or disappeared entirely) after incubation at elevated temperatures. The presence of KLUCEL thickener enabled the casting of consistent films. Films from blended formulations without KLUCEL thickener were not cast with any consistency.

Two blended adhesive and immunogically-active protein compositions were studied: KLUCEL thickener with sucrose or trehalose. The modified EUDRAGIT adhesive was blended with the other ingredients (PBS buffer with LT, disaccharide, and KLUCEL thickener) in a mass ratio of about 1:1.3. Final concentrations in the wet blend of KLUCEL thickener and disaccharide were 2.6% and 5%, respectively, and about 450 μg/gm LT. Patches from each blend composition were incubated at 5° C., room temperature, 40° C. or 60° C.

Chromatograms of the elution profile from RP-HPLC for protein extracted from each sample (i.e., KLUCEL thickener-pressure sensitive adhesive formulation with either sucrose or trehalose incubated at the four different temperatures) were analyzed. Changes in peak area ratios and normalized peak areas with incubation time were plotted. When 5% (w/v) disaccharide was included, the stability of LT was enhanced relative to the conditions in which little or no disaccharide is present, even at 40° C. Trehalose is a better stabilizer than sucrose under these conditions. At 5° C., protein was stabilized by both disaccharides over the incubation time tested. But LT was not stabilized at 60° C.; no LT was recovered after one week at this temperature.

Five blend compositions were studied with the modified EUDRAGIT adhesive: KLUCEL thickener and no disaccharide, KLUCEL thickener and sucrose, KLUCEL thickener and trehalose, NATROSOL thickener and sucrose, and NATROSOL thickener and trehalose. For patches containing KLUCEL thickener, the modified EUDRAGIT adhesive was blended with the other components (PBS buffer with LT, with or without disaccharide and KLUCEL thickener) at a mass ratio of about 1:1. For patches containing NATROSOL thickener, it was blended in a mass ratio of about 1:1.4. Final concentrations in the wet blend of thickener and disaccharide (if present) were 3% and 0.4%, respectively for KLUCEL patches, and 3.5% and 0.3%, respectively for NATROSOL patches. Final concentration of LT in the wet blend was about 500 μg/gm. Patches from each of the five blends were incubated at room temperature, 40° C. or 60° C.

Chromatograms of the elution profile from RP-HPLC for protein extracted from each sample (i.e., KLUCEL thickener or NATROSOL thickener-pressure sensitive adhesive formulation incubated at the three different temperatures) were analyzed. Changes in peak area ratios and normalized peak areas with incubation time were plotted. When 5% (w/v) disaccharide was included, the stability of LT was significantly enhanced relative to the conditions in which little or no disaccharide was present. Thickeners are used to enhance the viscosity of a formulation component so it can be cast as a uniform film. KLUCEL thickener is preferred to NATROSOL thickener under these conditions, because LT appears to be more stable in the presence of KLUCEL thickener than NATROSOL thickener. At low concentrations of disaccharide (i.e., 0.3% to 0.4%), no additional stability appears to be conferred to LT after one week of incubation. Therefore, higher disaccharide concentrations are preferred.

An adhesive-protein formulation further containing about 3% (w/v) thickener (e.g., KLUCEL hydroxypropyl cellulose) and about 5% (w/v) nonreducing sugar (e.g., trehalose) is preferred. A period of curing at an elevated temperature (40° C. to 60° C.) might be used to address any problem of crumbling and flakiness of at least partially-dried films. It may also be possible to include glycols at concentrations low enough (e.g., 1% or less of glycerol and/or 1,3-butanediol in the final wet blend may be used as a starting point, up to about 5%, 10% or 15%) not to destabilize the protein but sufficient to confer malleability and cohesion to the partially-dried pressure-sensitive adhesive layer. Thin films prepared by casting and drying wet blends of standard EUDRAGIT adhesive and buffers containing 3% (w/v) to 5% (w/v) disaccharide have a tendency to flake off the backing material used for patches and do not have much adhesive character. To improve malleability and adhesiveness, the standard EUDRAGIT adhesive may be modified by adding glycerol (up to about 6%) and 1,3-butanediol (up to about 4%). Addition of these plasticizers achieve the desired effect in terms of malleability and adhesiveness, but they may also be detrimental to LT stability.

Patches cast from a blend of standard EUDRAGIT adhesive and protein in a buffer containing disaccharide resulted in very inconsistent coat weights from patch to patch. It was found that including about 3% (w/w) KLUCEL or NATROSOL thickener in the final wet blend greatly increased the ability to cast consistent coats.

Patch compositions have been identified in which LT shows good stability and recoverability. Wet blended formulations contain about 500 μg/gm LT, 5% (w/v) disaccharide (e.g., sucrose, trehalose), and 3% (w/v) KLUCEL thickener. The blend was prepared by mixing EUDRAGIT adhesive and buffered protein solution (pH 7.4; containing disaccharide and KLUCEL thickener) at a mass ratio of about 1:1. There was excellent stability at 5° C. There was good stability at room temperature so we observed over the course of 6 to 7 weeks.

Addition of a stabilizer, which was a disaccharide (e.g., sucrose, trehalose), at a high concentration of 5% (w/v) may protect against aggregation, degradation, and denaturation. This structural stability is correlated to retention of biological activity. Trehalose appears to confer slightly more stability than sucrose, but lactose is detrimental to LT stability in patches. Excipients such as glycerol and 1,3-butanediol may also be somewhat detrimental to LT stability in an at least partially-dried patch.

The presence of lactose in the LT and LTR192G formulations from commercial suppliers also has a deleterious effect on solubility. LT formulated in lactose (such as that obtained from SSVI) is very poorly soluble in lactose-free solutions. Additionally, lactose may chemically modify a protein as indicated by mass spectrometry results showing a 14 amu difference in fragments generated from the lactose-formulated LT relative to the lactose-free LT adhesive formulation.

Adding KLUCEL thickener allowed the casting of consistently uniform films using a knife. Increasing concentrations of disaccharide and KLUCEL thickener increasingly caused the film to be brittle or flaky and to lose adhesive properties. Modification of EUDRAGIT adhesive by including excipients like glycerol and 1,3-butanediol (in concentrations of around 3% and 2%, respectively, in the final wet blend) restore the malleability and much of the adhesiveness of the film. These additions, however, are detrimental to protein stability. Incubation at elevated temperature (40° C.) for about a week, showed a restoration of malleability and film cohesiveness. This suggests that “curing” films for a short period of a few hours or less at elevated temperatures (40° C. to 50° C.) may be a viable means of restoring film malleability and integrity without having to add excipients harmful to protein stability. Freshly prepared EUDRAGIT adhesive should be used to avoid excessive crosslinking between polymers.

LT-in-Adhesive Formulation for Transcutaneous Immunization

The following aqueous-based adhesive was used for the pressure-sensitive adhesive layer. An acrylate adhesive is blended with acetyl-tributyl citrate (ATBC) as plasticizer and succinic acid as tackifier.

TABLE 1
Adhesive Formulation

| | Nonvolatile | Wet Weight | | Dry Weight | |
|---|---|---|---|---|---|
| | Component | weight | | weight | |
| Ingredients | % NVC | (gm) | % | (gm) | % |
| Methacrylate | 100 | 22.8 | 22.0 | 22.8 | 58.8 |
| Polymer | | | | | |
| Succinic Acid | 100 | 1 | 0.96 | 1 | 2.58 |
| ATBC | 100 | 15 | 14.5 | 15 | 38.7 |
| Water | 0 | 65 | 62.6 | 0 | 0 |
| Total | | 103.8 | 100 | 38.8 | 100 |

Dry weight % is the total weight of the component multiplied by % NVC divided by the total weight of all components multiplied by their respective % NVC. This adhesive formulation is used to make an emulsion containing protein.

TABLE 2
Adjuvant-in-Adhesive Formulation

| | Nonvolatile | Wet Weight | | Dry Weight | |
|---|---|---|---|---|---|
| | Component | weight | | weight | |
| Ingredients | % NVC | (gm) | % | (gm) | % |
| 1 × PBS/Lactose | 6.05 | 23.4 | 38.8 | 1.42 | 13.3 |
| Adhesive Formulation | 37.5 | 23.4 | 38.8 | 8.78 | 82.8 |
| NATROSOL thickener | 2.5 | 13.5 | 22.4 | 0.338 | 3.18 |
| LT protein adjuvant | N/A | 0.0234 | 0.04 | 0.023 | 0.22 |
| Tween 20 | 100 | 0.05 | 0.08 | 0.05 | 0.47 |

Five blends were made using the adjuvant-in-adhesive formulation:

Blend 1 was as shown in Table 2.

Blend 2 included glycerol (5.4% dry weight).

Blend 3 included 1,3 butanediol (5.4% dry weight).

Blend 4 substituted KLUCEL thickener for NATROSOL thickener.

Blends 5 and 6 were Blend 1 applied by rotogravure and laminated to pressure sensitive acrylate or silicone adhesive layer.

TABLE 3
Adjuvant/Co-Administered
Antigen-in-Adhesive Formulation

| | Nonvolatile | Wet Weight | | Dry Weight | |
|---|---|---|---|---|---|
| | Component | weight | | weight | |
| Ingredients | % NVC | (gm) | % | (gm) | % |
| 1 × PBS/Lactose | 6.05 | 15.6 | 38.7 | 0.94 | 13.3 |
| EUDRAGIT EPO | 37.5 | 15.6 | 38.7 | 5.85 | 82.3 |
| NATROSOL thickener | 2.5 | 9.0 | 22.3 | 0.23 | 3.16 |
| CS6 protein antigen | 100 | 0.0468 | 0.11 | 0.047 | 0.66 |
| LT protein adjuvant | 100 | 0.0156 | 0.04 | 0.016 | 0.22 |
| Tween 20 | 100 | 0.03 | 0.07 | 0.03 | 0.42 |

Two blends were made using the adjuvant-in-adhesive formulation:

Blend 7 was as shown in Table 3.

Blend 8 was a 1:1 mixture of 26.6 mg/ml CS6 and Blend 3 applied by rotogravure and laminated to draw down of LT alone.

The following formulations were made:

A LT formulated in EUDRAGIT EPO adhesive/3.2% NATROSOL thickener/0.5% Tween

B LT formulated in EUDRAGIT EPO adhesive/3.0% NATROSOL thickener/5% glycerol/0.4% Tween

C LT formulated in EUDRAGIT EPO adhesive/3.2% NATROSOL thickener/5% 1,3 butanediol/0.4% Tween

D LT formulated in EUDRAGIT EPO adhesive/3.2% KLUCEL thickener/0.5% Tween

E LT and CS6 formulated in EUDRAGIT EPO adhesive/3.2% NATROSOL thickener/0.4% Tween

Chemical stability of formulations A to E were determined by reverse phase HPLC and physical stability was determined size exclusion HPLC. Reverse phase chromatography separates protein according to binding affinity and allowed detection of fragments that result from protein degradation. Size exclusion chromatography separates protein according to passage through pores and allowed detection of aggregates, dissociated subunits, precipitates, and unfolded polypeptide chains. Samples were stored at 15° C., 25° C. or 40° C. for one week. Dissociation of the LT-B subunit (a pentamer) from the LT-A subunit was only detected with formulation B.

Mice were transcutaneously immunized as described previously (Scharton-Kersten et al., Infect. Immun., 68:5306-5313, 2000). Briefly, the animals were shaved on the dorsum with a No. 40 clipper, which leaves no visible irritation or changes in the skin, and rested for 48 hr. Mice were anesthetized intramuscularly (IM) in the hind thigh or intraperitoneally (IP) with a ketamine/xylazine mixture during the immunization procedure to prevent self-grooming. The exposed skin surface was hydrated with an aqueous solution of 10% glycerol, 70% isopropyl alcohol, and 20% water; the stratum corneum was at least partially disrupted with sandpaper. A 1 cm² patch with 10 μg/cm² protein was applied epicutaneously for 24 hr with an adhesive tape placed over the patch to secure it on the animal. After removal of the patch, the animals were extensively washed, tails down, under running tap water for about 30 sec, patted dry, and washed again.

Induction of an antigen-specific immune response was assayed by ELISA of antibody against LT or CS6. IMMULON-2 polystyrene plates (Dynex Laboratories) were coated with 0.1 μg/well of antigen, incubated at room temperature overnight, blocked with a 0.5% casein buffer in PBS, washed, serial dilutions of specimen applied, and the plates incubated for 2 hr at room temperature. IgG (H+L) antibody was detected using HRP-linked goat anti-mouse IgG (H+L) (Biorad) for 1 hr. Bound antibody was revealed using 2,2′-azino-di (3-ethylbenzthiazoline sulphonic acid) substrate (ABTS; Kirkegaard and Perry) and the reaction stopped after 30 min using a 1% SDS solution. Plates were read at 405 nm. Antibody titer results are reported in either OD (405 nm) or ELISA Units, which are defined as the inverse dilution of the sera that yields an optical density (OD) of 1.0.

TABLE 4
ELISA Results for Immunized Mice

| | | Geometric Mean |
|---|---|---|
| Formulation | Dose | of ELISA Units |
| A | 10 μg/cm² | 3325 |
| B | 10 μg/cm² | 6962 |
| C | 10 μg/cm² | 4896 |
| D | 10 μg/cm² | 12,707 |
| E | 10 μg/cm² (LT) | 9959 |
| | 30 μg/cm² (CS6) | 614 |
| Gauze Patch (LT) | 10 μg | 4861 |
| Gauze Patch (LT) | 10 μg | 3109 |
| Gauze (-) control | PBS | 12 |

Gauze patches were produced by adding an LT-containing solution to a 1 cm² Nu-Gauze backing layer. The substrate is held in place on the mouse with a piece of adhesive tape.

Protein-in-Adhesive Formulation for Transcutaneous Immunization

These protein-in-adhesive formulations are intended to treat enterotoxigenic E. coli (ETEC) by incorporating one or more ETEC subunit antigens into an adhesive formulation. The formula is also suitable for incorporating killed ETEC whole cells (˜10⁴ to 10⁸ killed bacteria per dose) with or without LT-adjuvant. The blend is then cast over a sheet of occlusive (or semi-occlusive) backing as a thin film. The formulation is allowed to cure (room temperature or 40° C. to 60° C.) until the film is at least partially-dried (water content may vary between 0.5% and 5%; less than about 1% or 2% is preferred for a patch according to the invention). The cast film may be cut from the die-cast to the desired size and shape. The patch may then be sealed in a light-tight, waterproof plastic or metal foil pouch. Patches produced in this manner may be stored refrigerated or at ambient temperatures (e.g., 20° C. to 30° C.). The protein-in-adhesive is flexible in that the multivalent vaccine blend may be varied to incorporate different amounts and ratios of one or multiple antigens and adjuvant. In addition, the patch size may be varied in order to adjust dosing. Depending upon the age of the individual, patch size (dose) can be varied for use in children and adults.

Protein-in-adhesive formulations are flexible and uniquely allow the vaccines to be coated in layers. These patches are manufactured in a manner wherein each vaccine component is layered separately onto the patch backing. The objective is to create a multilaminar membrane in which an adhesive formulation is adhered onto the backing layer, a first immunogenic formulation is applied on the adhesive formulation, a second immunogenic formulation is applied on the first immunogenic and adhesive formulation, and the release liner is the layer most distal with respect to the backing layer. The advantage of this approach is that it provides flexibility to the formulation (ie., a patch may be produced from the same process using different ratios of antigen-containing first immunogenic formulation and adjuvant-containing second immunogenic formulation, or where a patch is manufactured to contain only one or two active ingredients). The multilaminated patch also has the advantage of controlling the release rates of each antigen and the adjuvant. In some instances, it will be desirable to have LT adjuvant released immediately in order to pre-prime the skin dentritic cells (e.g., Langerhans cells) prior to release of other antigens. Then the LT-primed Langerhans cells may more efficiently capture and process the toxin and colonization factor antigens. Controlled delivery is a more efficient use of the adjuvant and antigens and will allow the doses to be further reduced.

Formulations are described that may be suitable for stabilizing proteins with at least adjuvant and/or antigen activity in contact with an adhesive. The following are intended to be examples of such formulations and are not intend to restrict the formulation.

Gel Formulations for Delivery of ETEC Subunit Vaccines (CS3, CS6, CFA/I, ST and LT) and Killed ETEC Whole Cells

Gels are examples of fully hydrated or wet patches. These formulations are intended to incorporate one or more ETEC subunit antigens entrapped within a gel matrix. This formulation is also suitable for transcutaneous delivery of killed ETEC whole cells (˜10⁴ to 10⁸ killed bacteria per dose) with or without LT. The vaccines are formulated by blending a solution containing the antigens in the desired amounts and ratios with Carbomer, Pluronic, or a mixture of the two gel components (see below). The gel-containing immunogenic formulation is then coated on a strip that holds the gel in place without spilling. It is important that the material have a low binding capacity for the proteins in the formulation. The strip may comprise patch materials as described above. The strip may be a single layer or a laminate of more than one layer. Generally, the strip is substantially water impermeable and helps to maintain the skin in hydrated condition. The material may be any type of polymer that meets the required flexibility and low binding capacity for proteins. Preferred polymers include, but are not limited to, polyethylene, ethyl vinylacetate, ethylvinyl alcohol, polyesters, or Teflon. The strip of material for holding the gel is less than 1 mm thick, preferably less than 0.05 mm thick, most preferably 0.001 to 0.03 mm thick.

The gel-loaded strip may be of different sizes and shapes. It is preferred that the corners be rounded for ease of application. The length of the strip can vary and is dependent upon the intended user (i.e., children or adults). It may be from about 2 cm to about 12 cm, and is preferably from about 4 cm to about 9 cm. The width of the strip will vary but it may be from about 0.5 cm to about 4 cm. The strip may have shallow pockets or dimples as reservoirs for the gel. To hold in place, when the gel-containing formulation is coated onto the strip, the gel should fill the reservoirs. The shallow pockets may be about 0.4 mm across and about 0.1 mm deep. The gel-loaded patch is about 1 mm thick, with a preferred thickness of about 0.5 mm or less. The gel-loaded strip is held in place by adhering it to a pressure-sensitive adhesive layer with the gel surface facing away from the adhesive. The backing material may be occlusive or semi-occlusive (e.g., TEGADERM dressing).

The flexural stiffness is important since maximal contact between the gel and the skin must be maintained. The strip will need to conform to the contour of the anatomical location where the patch is applied (e.g., skin over the deltoid muscle, volar forearm, neck, behind the ear, or other locations). Flexural stiffness can be measured with a Handle-O-Meter (Thwing Albert Instruments). The flexural stiffness should be less than 5 gm/cm, more preferably less than 3 gm/cm. The relatively low stiffness enables the strip of material to drape over the contoured surface with little force being exerted. The backing layer is designed to hold the patch in place, to aid in maintaining maximal contact between the skin and gel, and to prevent the gel from dehydrating during wear.

To prevent dehydration of the wet patch during storage and handling, it may be placed on an inert plastic strip, which is fairly rigid. The gel surface would be in direct contact with the plastic strip, and the gel/plastic interface has low peel force making it easy to separate the gel strip from the plastic strip. The plastic strip may be made of polyethylene or similar material. The gel-containing patch can be packaged in a light-proof and water tight plastic or foil pouch. The pouch can be stored at room temperature or in a refrigerator.

The following are intended as examples of the hydrated gel formulation and are not intended to restrict it: gels in phosphate buffered saline; 1% Carbomer 1342; 1.5% Carbomer 940; 1.5% Carbomer 934; 1.5% Carbomer 940, 2% sucrose, 10% isopropyl alcohol, 10% glycerol; 50% Pluronic F87; and 30% Pluronic F108.

Carbomer polymers are high molecular weight, acrylic acid-based polymers that may be cross-linked with allyl sucrose or allylpentaerythritol, and/or modified with C10-C30 alkyl acrylates. These may or may or not be incorporated into a patch or may be delivered by other means know in the art into the skin.

Formulations may be comprised of carbomers of different average molecular weights. For example, the polymers may be Carbomer 1342 (e.g., 1% Carbomer 1342, 0.6 mg/ml LT, 0.3% methylparaben, 0.1% propylparaben, 2.5% lactose, in 1×PBS); Carbomer 934 (e.g., 1.5% Carbomer 934, 0.6 mg/ml LT, 0.3% methylparaben, 0.1% propylparaben, 2.5% lactose, in 1×PBS); or Carbomer 940 (e.g., 1.5% Carbomer 940, 0.6 mg/ml LT, 0.3% methylparaben, 0.1% propylparaben, 2.5% lactose, in 1×PBS). Each formulation can be prepared in a phosphate buffered saline solution and contain LT at a concentration of about 0.6 mg/ml or less, but antigens and adjuvants may also be formulated from about 0.001 mg/ml to about 0.6 mg/ml or from about 0.6 mg/ml to about 6 mg/ml. In addition, antimicrobial agents such as methylparaben and propylparaben may be included.

Combinations of Carbomer 940 and Pluronic F87 (e.g., 1.5% Carbomer 940, 0.5% Pluronic F87, 0.6 mg/ml LT, 0.3% methylparaben, 0.1% propylparaben, 2.5% lactose, in 1×PBS) may be used. Pluronics are another class of hydrogel that contain repeating segments of ethylene oxide-propylene oxide-ethylene oxide. The amount of LT and antimicrobial agents in the formulation may be identical.

Other formulations may enhance delivery using penetration enhancers and carbomers. For example, a gel may comprise Carbomer 940 with Pharmasolve (e.g., 1.5% Carbomer 940, 10% Pharmasolve, 0.6 mg/ml LT, 0.3% methylparaben, 0.1% propylparaben, 2.5% lactose, in 1×PBS) while the final gel may contain Carbomer 940, glycerol, and isopropanol (e.g., 1.5% Carbomer 940, 10% glycerol, 10% isopropanol, 0.6 mg/ml LT, 0.3% methylparaben, 0.1% propylparaben, 2.5% lactose, in 1×PBS). The concentration of LT and antimicrobial agents may remain identical to the above formulations, or may be in other ranges specified.

All references (e.g., articles, books, patents, and patent applications) cited above are indicative of the level of skill in the art and are incorporated by reference.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. No particular relationship between or among limitations of a claim is meant unless such relationship is explicitly recited in the claim (e.g., the arrangement of components in a product claim or order of steps in a method claim is not a limitation of the claim unless explicitly stated to be so). Thus, all possible combinations and permutations of the individual elements disclosed herein are intended to be considered part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification

We claim:

1. A patch for transcutaneous immunization comprising at least four different components:
(a) a backing layer,
(b) a pressure-sensitive adhesive layer adhering to the backing layer, and an immunogenic formulation applied to and/or incorporated in the pressure-sensitive adhesive layer comprising:
(c) at least one protein in contact with adhesive of the pressure-sensitive adhesive layer, wherein the at least one protein is immunologically active and
(d) a stabilizer which maintains the immunological activity of the at least one protein in the adhesive's presence;
wherein the patch is epicutaneously applied to a subject's skin with the pressure-sensitive adhesive layer adhering to the skin and the backing layer distal thereto, such that an effective amount of the at least one protein induces an antigen-specific immune response in the subject by transcutaneous immunization.

2. The patch according to claim 1, wherein the backing layer is occlusive.

3. The patch according to claim 1, wherein the adhesive is an aqueous-based adhesive.

4. The patch according to claim 1, wherein the adhesive is an acrylate adhesive.

5. The patch according to claim 1, wherein the at least one protein has adjuvant activity.

6. The patch according to claim 1, wherein the at least one protein is an ADP-ribosylating exotoxin, a fragment thereof, or a mutant thereof.

7. The patch according to claim 1, wherein the at least one protein is an E. coli heat-labile exotoxin, a fragment thereof, or a mutant thereof.

8. The patch according to claim 1, wherein the at least one protein is the antigen against which the antigen-specific immune response is induced.

9. The patch according to claim 1, wherein there is between 1 μg and 100 μg of the least one protein.

10. The patch according to claim 1, wherein the stabilizer is a nonreducing sugar.

11. The patch according to claim 1, wherein the stabilizer is sucrose or trehalose.

12. The patch according to claim 1 further comprising a fifth component (e) a release liner, wherein the pressure-sensitive adhesive layer is positioned between the backing layer and the release liner such that peeling the release liner exposes the pressure-sensitive adhesive layer and allows the patch to be epicutaneously applied to the subject's skin with the backing layer distal thereto.

13. The patch according to claim 1, wherein a single patch is packaged such that the at least one protein is effective to induce the antigen-specific immune response in the subject by transcutaneous immunization for at least two years.

14. The patch according to claim 1, wherein the pressure-sensitive adhesive layer further comprises at least one plasticizer and at least one tackifier.

15. The patch according to claim 14, wherein the plasticizer is a trialkyl citrate.

16. The patch according to claim 14, wherein the tackifier is one or more glycols and/or succinic acid.

17. The patch according to claim 1, wherein the immunogenic formulation further comprises a thickener.

18. The patch according to claim 17, wherein the thickener is a hydroxyalkyl cellulose or starch.

19. Use of the patch according to any one of claims 1-18 to induce an antigen-specific immune response.

20. Use of the patch according to any one of claims 1-18 to treat and/or prevent one or more symptoms associated with disease.

21. The use according to claim 19 or 20 further comprising hydrating the skin prior to application of the patch.

22. The use according to claim 19 or 20 further comprising enhancing penetration of the skin by chemical and/or physical energy to disruption stratum corneum without perforating dermis of the skin prior to application of the patch.

23. Method for manufacturing a patch according to any one of claims 1-18 intended for transcutaneous immunization, characterized in that an immunogenic formulation comprising (a) immunogen comprising at least one immunologically-active protein and (b) stabilizer which maintains the immunological activity of the at least one protein in a suspension or solution with pressure-sensitive adhesive is applied to and/or incorporated in a pressure-sensitive adhesive layer, wherein the pressure-sensitive adhesive layer is adhered to a backing layer.

24. A formulation comprising (a) pressure-sensitive adhesive, (b) immunogen comprising at least one immunologically-active protein, and (c) stabilizer which maintains the immunological activity of the at least one protein in a suspension or solution with the pressure-sensitive adhesive.

25. Use of the formulation according to claim 24 to manufacture a patch for transcutaneous immunization by adhering the formulation to a backing layer.
